(19) United States
(12) Patent Application Publication
(10) Pub. No.: US 2006/0165667 A1
(43) Pub. Date: Jul. 27, 2006

(54) Novel methods, compositions and devices for inducing neovascularization

(75) Inventors: Mary J. Laughlin, Shaker Heights, OH (US); Vincent Pompili, Hudson, OH (US)

Correspondence Address: FISH & NEAVE IP GROUP;ROPES & GRAY LLP, ONE INTERNATIONAL PLACE, BOSTON, MA, 02110-2624, US

(73) Assignee: Case Western Reserve University, Cleveland, OH (US)

(21) Appl. No.: 11/295,311

(22) Filed: Dec. 5, 2005

Related U.S. Application Data
(60) Provisional application No. 60/633,292, filed on Dec. 3, 2004.

(51) Int. Cl. A61K 48/00; C12N 5/08

(52) U.S. Cl. 424/93.21; 435/372

(57) ABSTRACT

The invention provides methods of inducing neovascularization in a subject in need thereof. The invention further provides compositions, devices and implantable products generated from conditioned media, and in particular, from conditioned media from cultured umbilical cord populations. These compositions are useful for inducing neovascularization. The invention also provides methods of distributing compositions, devices and products to health care professionals.

### CROSS-REFERENCE TO RELATED APPLICATIONS

This application claims the benefit of the filing date of U.S. Application No. 60/633292, filed Dec. 3, 2004, entitled “NOVEL METHODS, COMPOSITIONS AND DEVICES FOR INDUCING NEOVASCULARIZATION.” The entire teachings of the referenced application are incorporated by reference herein.

### GOVERNMENT SUPPORT

The invention described herein was supported, in whole or in part, by grant 1R21-HL-72362-01 from the National Institutes of Health. The United States government has certain rights to the invention.

### BACKGROUND OF THE INVENTION

Atherosclerotic cardiovascular disease is a leading cause of morbidity and mortality in the industrialized western hemisphere. Coronary artery disease, the pathologic process of arterial luminal narrowing by atherosclerotic plaque resulting in obstruction of blood flow to the heart, accounts for about half of the deaths. Peripheral vascular occlusive disease and its complications, including ulcers and even necrosis of the affected limb, are also common. Although catheter-based revascularization or surgery-based treatment approaches have been successful in restoring blood flow to ischemic myocardium in the majority of cases, the treatments are inadequate for a significant number of patients who remain incompletely revascularized. The ramifications of treatment limitations may be significant in patients who have large areas of ischemic, but viable myocardium jeopardized by the impaired perfusion supplied by vessels that are poor targets for conventional revascularization techniques.

Treatment alternatives, including mechanical approaches such as percutaneous transluminal myocardial revascularization, and the like, have not produced encouraging results. Gene therapy using adenoviral vectors to augment cytokine production and, therefore, promote angiogenesis has shown promise, but this therapy has limitations and has not yet emerged as the optimal treatment for these patients. Therefore, therapeutic angiogenesis has attracted many researchers attempting to discover a way to circumvent the burden of chronic myocardial ischemia.

Therefore, there is still a need to develop treatment modalities for both myocardial ischemia and peripheral vascular disease that can promote vasculogenesis in the ischemic tissue. The present invention provides novel methods, compositions and devices, such as implantable devices, which may be used in therapies for inducing neovascularization.

### SUMMARY OF THE INVENTION

One aspect of the invention provides compositions comprising cell culture medium (or components thereof) conditioned by cells grown in culture, preferably umbilical cord blood cells. The cells may be grown under serum-free conditions supplemented with growth factors. The cells used to condition the medium may be genetically modified to alter the concentration of proteins found in the medium. The conditioned cell medium may be processed for various applications, which include pharmaceutical applications. The invention also relates to compositions containing extracellular matrix proteins and/or other purified protein(s) derived from the conditioned medium. The invention further provides formulations and devices for the delivery of the conditioned media components.

A related aspect of the invention provides a method of distributing any one of the conditioned media compositions, devices and products described herein, for use by health care professionals. The invention further provides methods of providing therapeutic compositions derived from umbilical cord blood for use by health care professionals for the treatment of a disorder in a subject.

The invention further provides agents for the manufacture of medicaments, compositions or devices to treat any of the disorders described herein, including for treating ischemia or for inducing neovascularization. Any methods disclosed herein for treating or preventing a disorder by administering an agent to a subject may be applied to the use of the agent in the manufacture of a medicament to treat that disorder. For example, in one specific embodiment, the protein components of conditioned media from umbilical cord AC133+ cells may be used in the manufacture of a medicament for the treatment of cardiac or peripheral ischemia.

### BRIEF DESCRIPTION OF THE DRAWINGS

FIGS. 1A-1C illustrate fluorescent cytochemical staining of endothelial precursor cells EPC derived from umbilical cord blood (UCB) under short-term endothelial-driving culture conditions. Panel A illustrates uptake of acetylated low-density lipoprotein (acLDL). Panel B illustrates adherence of Ulex europaeus agglutinin (UEA-1). Panel C illustrates composite dual staining for acLDL and UEA-1. Images were recorded using a confocal microscope at 40× magnification.

FIG. 2 illustrates staining of EPC derived from UCB for von Willebrand factor (vWF) and also illustrates the spindle-like morphology characteristic of EPCs. The cells were studied using phase contrast microscopy using a 40× magnification. Brown perinuclear stain is due to immunoperoxidase conjugated to secondary antibodies that reacted with perinuclear vWF particles.

FIG. 3 illustrates flow cytometry analysis of the surface phenotype of CD133⁺ cells selected from UCB. FSC gain was increased for better resolution of very small cells. Distinct populations of CD133⁺/CD34⁻ cells (100) and CD133⁺/CD34⁺ cells (200) were identified. No gating was applied.

FIG. 4 illustrates flow cytometry analysis of a comparison of endothelial cell characteristics of EPC cells derived from UCB and human bone marrow (BM) after 19 days and 12 days of culture in endothelial-driving culture conditions. Adherent cells were trypsinized and stained for CD34 and endothelial-specific markers VE-cadherin, CD146 and CD31. The non-stained control is shown in black. The stained cells are shown in gray.

FIG. 5 depicts the results of neovascularization achieved by transplantation of UCB- and BM-derived EPC into an in vivo mouse hind-limb ischemia model. NOD/SCID mice underwent femoral artery ligation and excision followed by injection of saline, medium or cells cultured for 7 days in endothelial-driving culture conditions. Laser Doppler measurements were taken post-op and then every week under the same conditions. Depicted is a comparison of the perfusion ratio between the ischemic and non-ischemic let.

FIGS. 6A-6B illustrate a histological assessment of the ischemic hind limb at 28 days after surgery. The hind limb of the ischemic leg of the mouse injected with UCB-derived EPC showed positive CD31 staining, indicated by the white arrows (Panel A). The control mouse, injected with medium only, was negative for CD31 (Panel B).

FIG. 7 illustrates the results of isolation and purification of CD133⁺ cells from UCB. Mononuclear cells (MNC) were labeled with anti-CD133 conjugated magnetic beads, followed by automated sorting through magnetic columns (Automacs, Miltenyi). The yield of the labeled CD133⁺ cells after passage through one magnetic column was routinely about 0.4% of the MNC cells, with a purity ranging between 75% and 85% (83.02% illustrated). After staining with CD133-PE, the cells were FACS sorted for PE fluorescence, raising the purity to 98.87%, with a final yield of 0.1% of the initial MNC input. No gating was applied.

FIG. 8 illustrates differential expression of CD45, CD34, BCL-2 and p21 in purified CD133⁺ cells after 24 hours of culture under hematopoietic-driving or endothelial-driving conditions. The percentages are of the total cells analyzed.

FIGS. 9A-9C illustrate a cell cycle analysis in cultured purified CD133⁺ cells. The CD133⁺ cells were purified and analyzed for cell cycle stages (A) immediately; (B) cultured for 24 hours under hematopoietic-driving or endothelial-driving conditions; or (C) cultured for 72 hours under hematopoietic-driving conditions. Cells were fixed, permeabilized, the DNA stained with Hoechst, and analyzed for cell cycle stages.

FIG. 10 depicts neovascularization by EPC derived from purified CD133⁺ cells in the mouse hind-limb ischemia model. Blood flow was measured over time by Laser Doppler and expressed as the ratio between the ischemic and non-ischemic leg.

FIGS. 11A-11D illustrate the dose response mitotic expansion of human mesenchymal stem cell (hMSC) number following incubation in medium conditioned by human umbilical vein endothelial cells (HUVECs) (B), and the dose response mitotic expansion of HUVEC cell number following incubation in medium conditioned by hMSCs (C). (A) and (D) are control growth cultures.

FIG. 12 illustrates migration of hMSCs (top) and HUVECs (bottom) toward hMSC-conditioned medium (left) and migration of HUVECs (top) and hMSCs (bottom) toward HUVEC-conditioned medium (right).

FIG. 13 illustrates that hMSCs express vascular endothelial growth factor (VEGF) genes. The expression of VEGF family growth factor mRNA was determined using RT-PCR. Specific primers were added to cDNA to amplify VEGF family genes over 35 cycles. Varying amounts of PCR product were run on a 2% agarose gel and visualized using ethidium bromide staining. The size of the PCR products are as follows: VEGF-A at 577 bp, 526 bp, and 454 bp; VEGF-B at 326 bp and 225 bp; VEGF-C at 183 bp; VEGF-D at 225 bp; and PIGF at 248 bp and 184 bp.

FIG. 14 illustrates VEGF receptor mRNA expression by hMSCs. Total RNA was added to specific primers to amplify VEGF receptor genes by RT-PCR. Varying amounts of PCR product were run on a 2% agarose gel and visualized using ethidium bromide staining. Shown are high molecular weight DNA markers, VEGFR1 (1,098 bp); VEGFR2 (326 bp); VEGFR3 (380 bp); Neuropilin-1 (375 bp) and Neuropilin-2 (304 bp and 289 bp).

FIGS. 15A-15B illustrate ELISA analysis of active TGF-b1 in monocultured or co-cultured hMSCs and HUVECs. Monocultured hMSCs and HUVECs secrete latent TGF-b1 protein (A). Co-culture of hMSCs and HUVECs produces active TGF-b1 protein (B).

FIGS. 16A-16D illustrate that hMSCs selectively migrate to endothelial tube-like structures. HUVECs in monoculture (A) were induced to form tube-like structures by addition of Vitrogen gel (B). DiI stained hMSCs were added to the top of the gel cultures (C). 24 hours later, the hMSCs are located along endothelial cell tube-like structures (D).

FIG. 17 shows the release of angiogenic factors by CD133 cells. Protein secretion read-out was measured by angiogenic cytometric bead (CBA). Supernatants of cultured CD133+ and MNCs from UCD, as well as MSC for adult BM, were collected after 24 hour and secreted factors were measured.

FIGS. 18A-18B show that CD133+ EPC associate with HUVECs on Matrigel. CM-Dil-labeled CD133 EPC associate with developing HUVEC tubules after 24 hours. (A) Co-cultures observed under visible light or (B) under fluorescence light.

FIGS. 19A-B show HUVEC-stromal cell interactions in an organotypic culture system. Stromal cells were grown to confluency prior to being seeded with HUVEC alone (A) or with equivalent numbers of HUVEC and CD133+ EPC (B). After 2 weeks, cultures were fixed and permeabilized with 60% acetone/PBS and stained with anti-CD31-FITC antibody. CD133+ EPC enhanced both the formation of tubules and stimulated the proliferation of HUVEC.

FIG. 20 shows Mixed Lymphocyte Reactions (MLR) with and without allogeneic hMSC. Allogeneic T-cell activation was measured by IFNγ EliSpot generated in 11 independent experiments without and with addition of 11 different 3^{rd} party hMSC.

FIG. 21 shows that T-Cell inhibitory function of huMSC requires an activation step by blood MNC.

FIG. 22 shows that blood CD14⁺ monocytes activate huMSC to secrete soluble immunosuppressive factor(s):

FIG. 23 shows the Role of TGFβ in hMSC Mediated Inhibition of MLR.

### DETAILED DESCRIPTION OF THE INVENTION

I. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The term “expression vector” and equivalent terms are used herein to mean a vector which is capable of inducing the expression of DNA that has been cloned into it after transformation into a host cell. The cloned DNA is usually placed under the control of (i.e., operably linked to) certain regulatory sequences such a promoters or enhancers. Promoter sequences may be constitutive, inducible or repressible.

The term “operably linked” is used herein to mean molecular elements that are positioned in such a manner that enables them to carry out their normal functions. For example, a gene is operably linked to a promoter when its transcription is under the control of the promoter and, if the gene encodes a protein, such transcription produces the protein normally encoded by the gene. For example, a binding site for a transcriptional regulator is said to be operably linked to a promoter when transcription from the promoter is regulated by protein(s) binding to the binding site. Likewise, two protein domains are said to be operably linked in a protein when both domains are able to perform their normal functions. The term “encoding” comprises an RNA product resulting from transcription of a DNA molecule, a protein resulting from the translation of an RNA molecule, or a protein resulting from the transcription of a DNA molecule and the subsequent translation of the RNA product.

The term “promoter” is used herein to mean a DNA sequence that initiates the transcription of a gene. Promoters are typically found 5′ to the gene and located proximal to the start codon. If a promoter is of the inducible type, then the rate of transcription increases in response to an inducer. Promoters may be operably linked to DNA binding elements that serve as binding sites for transcriptional regulators. The term “mammalian promoter” is used herein to mean promoters that are active in mammalian cells. Similarly, “prokaryotic promoter” refers to promoters active in prokaryotic cells.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non homologous recombination.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The term “effective amount” refers to the amount of a therapeutic reagent that when administered to a subject by an appropriate dose and regime produces the desired result.

The term “subject in need of treatment for a disorder” is a subject diagnosed with that disorder or suspected of having that disorder.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically cleaved or recombinantly prepared portions of an antibody molecule that are capable of selectively reacting with a certain protein. Non limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or noncovalently linked to form antibodies having two or more binding sites. The term antibody also includes polyclonal, monoclonal, or other purified preparations of antibodies and recombinant antibodies.

The term “conditioned media” (also called “Conditioned Cell Media” or “Conditioned Cell and Tissue Culture Media”) as used herein refers to a formulation containing extracellular protein(s) and cellular metabolites, prepared by culturing a first population of cells in a medium, and then harvesting the medium.

A “growth environment” is an environment in which cells of interest will proliferate in vitro. Features of the environment include the medium in which the cells are cultured, the temperature, the partial pressure of O₂ and CO₂, and a supporting structure (such as a substrate on a solid surface) if present.

A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors.

A polymer, or polymer matrix, is “biocompatible” if the polymer, and any degradation products of the polymer, are substantially non-toxic to the recipient and also present no significant deleterious or untoward effects on the recipient's body, such as a significant immunological reaction at the site of administration.

Biodegradable, as defined herein, means the composition will degrade or erode in vivo to form smaller chemical species. Degradation can result, for example, by enzymatic, chemical and/or physical processes. Suitable biocompatible, biodegradable polymers include, for example, poly(lactides), poly(glycolides), poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetals, polycyanoacrylates, polyetheresters, poly(dioxanone)s, poly(alkylene alkylates)s, copolymers of polyethylene glycol and polyorthoester, biodegradable polyurethanes, blends and copolymers thereof.

The following abbreviations are used throughout the specification: BFU-E=burst-forming unit-erythroid, CFU-C=colony forming, unit-culture, CFU-GEMM=colony forming unit-granuloid, erythroid, monocyte, megakaryocyte, EDTA=ethylene diamine tetraacetic acid, FBS=fetal bovine serum, HBSS=Hank's balanced salt solution, HS=horse serum, LTBMC=long term bone marrow culture, MEM=minimal essential medium, PBL=peripheral blood leukocytes, PBS=phosphate buffered saline, RPMI 1640=Roswell Park Memorial Institute medium number 1640 (GIBCO, Inc., Grand Island, N.Y.).

Other technical terms used herein have their ordinary meaning in the art that they are used, as exemplified by a variety of technical dictionaries, such as the McGraw Hill Dictionary of Chemical Terms and the Stedman's Medical Dictionary.

II. Cells, Compositions and Formulations

One aspect of the invention provides composition comprising conditioned cell culture medium, or components isolated therefrom, from a population of cells. Such compositions are useful for the treatment of disorders, and in particular of disorders where neovascularization is beneficial.

In one embodiment, the composition comprises secreted polypeptides or conditioned medium from a cultured cell population, wherein at least 10%, 15%, 20%, 25%, 30%, 35%, 40%, 45%, 50%, 55%, 60%, 65%, 70%, 75%, 80%, 85%, 90%, 95%, 96%, 97%, 98% or about 99% of the cells which produce the conditioned media are selected from the group consisting of CD133⁺ cells, CD34⁻CD133⁻CD73⁻ cells, EPCs, MAPCs, mesenchymal stem cells, and combinations thereof. In one preferred embodiment, the population of cells is derived from umbilical cord blood, which may be from a single umbilical cord or from a plurality of umbilical cord. In one embodiments, the composition also includes a conditioned media component from a second population of cells that is different from the first population of cells. In one embodiment, the second population of cells comprises (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In one preferred embodiment, the second population of cells is derived from umbilical cord blood, which may be from a single umbilical cord or from a plurality of umbilical cords, and which may be from the same cord blood sample as the first population or from a different sample. In one embodiment, at least 5%, 10%, 20%, 30%, 40%, 50%, 60%, 70%, 80%, 90%, 95% or greater of the cells in the second population are (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In a preferred embodiment, the CD133+ cells in the second population are CD133+CD34+KDR-CXCR4-cells.

In one preferred embodiment, the cells from which the conditioned medium is derived are AC133+ cells. The cell surface marker AC133+ is also known as CD133. AC133+ cells are found in peripheral blood, bone marrow, fetal liver and umbilical cord blood (Yin et al. 1997. Blood 90(12):5002-12; Gehling et al., 2000, Blood. 95(10): 3106-12; Buhring et al. 1999. Ann NY Acad Sci 99 872: 25-39; Majka et al. 2000. Folia Histochem Cytobiol. 38:53-63). Antibodies which recognize the CD133 antigen are described in U.S. Pat. No. 5,843,633. Methods and sources of isolating CD133⁺ cells are described, for example, in Intemational PCT Application Nos. WO03/095631, WO99/37751, and WO01/94420; and U.S. Patent Publication Nos. 2003/0091547, 2003/0199464 and 2002/0051762, the entire teachings of which are herein incorporated by reference. In one embodiment, the population of AC133+ cells also displays the following marker profiles: CD34⁺ (75%-99%), KDR (VEGFR2) (0-10%), CD105 (15%-30%) and CXCR4(2%-15%).

In one preferred embodiment, the cells from which the conditioned medium is derived are endothelial precursor cells (EPCs). Endothelial precursor cells may be isolated by culturing AC133+ or CD34+ cells in a substrate. Mandel et al. (2001) Blood 98(11): 55b describes the generation and culturing of EPCs from umbilical-cord derived AC133+ cells. Kalka et al. (2000) Proc Natl Acad Sci USA 97(7): 3422-7 and Gehling et al. (2000) Blood 95(10): 3106-12 describe the isolation of EPCs from peripheral blood. Kawamoto et al. (2001) Circulation 103: 634-637 describes the isolation and culturing of EPCs form human peripheral blood mononuclear cells. Burger et al. Blood 2002 15;100(10):3527-35 describes CD34+ cells selected from bone marrow, umbilical cord blood and peripheral blood. Additional methods may be found in Yang et al. Zhonghua Yi Xue Za Zhi,” 2003, 83(16). In some embodiments, EPCs are generated by culturing CD34+ cells. CD34+ cells may be isolated using an anti-CD34+ antibody (Andrews et al. Blood. 1986; 68(5):1030-5).

In another preferred embodiment, the cells from which the conditioned medium is derived are mesenchymal stem cells (MSCs). Methods to isolate, culture-expand and phenotypically characterize hMSCs, as well as their multi-lineage developmental potential and capacity to regulate a variety of other developmental events including angiogenesis, are known in the art (Fleming, J E Jr. et al. Dev. Dyn. 212, 119-132 (1998); Barry F P et al. Biochem. Biophys. Res. Commun. 265, 134-139 (1999)). Although hMSCs are rare, comprising about 0.01-0.0001% of the total nucleated cells of bone marrow, methodology for their isolation and purification to homogeneity, and mitotic expansion in culture without loss of their stem cell potential is established (Haynesworth S E et al. Bone 13, 81-88 (1992)). Human adult MSC, although marrow-derived, do not express CD34 or CD45, but have been shown to express IL-6, -7, -8, -11, -12, -14, -15, M-CSF, flt-3 ligand (FL), and SCF in steady state, and do not express IL-3 and TGFβ. Exposure to dexamethasone results in decreased expression of LIF, IL-6 and IL-11 (Haynesworth S E et al. J. Cell Physiol. 166, 585-592 (1996)). Mesenchymal stem cells for use in the methods according to the invention can be isolated for example from peripheral blood, umbilical cord blood or from bone marrow. A method for preparing hMSC has been described in U.S. Pat. No. 5,486,359. In one preferred embodiment, the mesenchymal stem cells are isolated from umbilical cord blood, such as described by Erices et al. 2000 Br. J Haematol 109(1):235-42 or Covas et al. Braz J Med Biol Res. September 2003; 26(9): 1179-83

Several techniques are known for the rapid isolation of mesenchymal stem cells including, but not limited to, leucopheresis, density gradient fractionation, immunoselection, differential adhesion separation, and the like. For example, immunoselection can include isolation of a population of hMSCs using monoclonal antibodies raised against surface antigens expressed by bone marrow-derived hMSCs, i.e., SH2, SH3 or SH4, as described, for example, in U.S. Pat. No. 6,387,367. The SH2 antibody binds to endoglin (CD105), while SH3 and SH4 bind CD73. Further, these monoclonal antibodies provide effective probes which can be utilized for identifying, quantifying and purifying hMSC, regardless of their source in the body.

The hMSC for use in the methods according to the invention can be maintained in culture media which can be chemically defined serum free media or less preferably can be a “complete medium”, such as Dulbecco's Modified Eagles Medium supplemented with 10% serum (DMEM). Suitable chemically defined serum free media that may be used to culture human MSCs is described in U.S. Pat. No. 5,908,782 and in WO96/39487. Chemically defined medium comprises a minimum essential medium such as Iscove's Modified Dulbecco's Medium (IMDM), supplemented with human serum albumin, human Ex Cyte lipoprotein, transferrin, insulin, vitamins, essential and non-essential amino acids, sodium pyruvate, glutamine and a mitogen. These media stimulate mesenchymal stem cell growth without differentiation. Culture for about 2 weeks results in 10 to 14 doublings of the population of adherent cells. After plating the cells, removal of non-adherent cells by changes of medium every 3 to 4 days results in a highly purified culture of adherent cells that have retained their stem cell characteristics, and can be identified and quantified by their expression of cell surface antigens identified by monoclonal antibodies SH2, SH3 and/or SH4.

In one embodiment, the cells from which the conditioned medium is derived are multipotent adult progenitor cells (MAPCs). The isolation of MAPCs from human bone marrow, and methods of culturing these cells, is described in Morayma Reyes et al. (2002) J. Clin Invest, Vol. 109, Number 3, 337-346. Preferred methods of MAPC isolation are described in WO 01/11011 and WO02/064748, herein incorporated by reference. MAPCs can be isolated from multiple sources, including bone marrow, muscle, brain, spinal cord, blood or skin. To isolate MAPCs, bone marrow mononuclear cells can be derived from bone marrow aspirates, which can be obtained by standard means known to those of skill in the art (see, for example, Muschler, G. F., et al., J. Bone Joint Surg. Am. (1997) 79(11): 1699-709, Batinic, D., et al., Bone Marrow Transplant. (1990) 6(2): 103-7).

MAPCs are present within the bone marrow (or in other organs, such as liver and brain), but do not express the common leukocyte antigen CD45 or glycophorin-A (GlyA). The mixed population of cells can be subjected to a Ficoll Hypaque separation. Cells can then be subjected to negative selection using anti-CD45 and anti-Gly-A antibodies, depleting the population of CD45+ and GlyA+ cells, and recovering the remaining approximately 0.1% of marrow mononuclear cells. Cells can also be plated in fibronectin coated wells and cultured as described below for 2-4 weeks after which the cells are depleted of CD45+ and GlyA cells. Alternatively, positive selection can be employed to isolate cells using a combination of cell-specific markers, such as the leukemia inhibitory factor (LIF) receptor. Both positive and negative selection techniques are known to those of skill in the art, and numerous monoclonal and polyclonal. Antibodies suitable for negative selection purposes are also known in the art (see, for example, Leukocyte Typing V, Schlossman, et al., Eds. (1995) Oxford University Press) and are commercially available from a number of sources.

In one embodiment, the cells from which the conditioned medium is derived are CD34⁻CD133⁻CD73⁻ cells. In another embodiment, the CD34⁻CD133⁻CD73⁻ cells express CD14, CD11b and/or CXCR4. In another embodiment, the CD34⁻CD133⁻CD73⁻ cells express two of these three surface markers. In another embodiment, the CD34⁻CD133⁻ CD73⁻ cells express the surface markers KDR or CD105 or both. In a preferred embodiment, the CD34⁻CD133⁻CD73⁻ cells are CD34⁻CD133⁻CD73⁻CD14⁺CD11b⁺CXCR4⁺ cells. In yet another embodiment, the CD34⁻CD133⁻CD73⁻ cells express one or more of the following surface markers: intercellular adhesion molecule-1 (ICAM-1), monocyte chemotactic protein-1 (MAC-1), lymphocyte function-associated antigen-1 (LFA-1), CD29, CD55, CD44, or combinations thereof. In another embodiment, the cells are CD34⁻CD133⁻CD73⁻ cells are also CD45⁻. Preferred populations of CD34⁻CD133⁻CD73⁻ cells and methods for their isolation are described in copending application provisional 60/589941, incorporated by reference herein.

In one embodiment of the methods described herein, the CD34⁻CD133⁻CD73⁻ cells are capable of differentiating into stromal cells. In another embodiment, the CD34⁻CD133⁻CD73⁻ cells are capable of differentiating into cells which express platelet endothelial cell adhesion molecule-1 (PECAM1), transcription factor GATA-2, N-cadherin, vascular endothelial-cadherin, von Willebrand factor, or combinations thereof. In some embodiments, CD34⁻CD133⁻CD73⁻ cells are isolated from umbilical cord blood. In another embodiment, CD34⁻CD133⁻CD73⁻ cells are isolated from bone marrow, while in another embodiment they are isolated from peripheral blood. When a compositions comprising CD34⁻CD133⁻CD73⁻ cells is used in the methods described herein for administration to a subject, the CD34⁻CD133⁻CD74⁻ cells may be autologous, allogenic, or HLA-compatible with the subject. The cells may be isolated from the subject's own bone marrow, peripheral blood or even umbilical cord blood.

In one embodiment, the cells from which the conditioned medium is derived are umbilical cord blood lineage negative (LinNeg) stem cells. Such cells are described in Guckin C P et al., (2004) Exp Cell Res. May 1;295(2):350-9

In one embodiment, the cells from which the conditioned medium is derived are genetically-engineered cells. Such cells can be modified, for example, to express a desired protein(s) so that the concentration of the expressed protein(s) in the medium is optimized for the particular desired application. In accordance with the present invention, the cells and tissue cultures used to condition the medium may be engineered to express a target gene product which may impart a wide variety of functions, including but not limited to, improved properties in expressing proteins resembling physiological reactions, increased expression of a particular protein useful for a specific application, such as for inducing neovascularization or for inhibiting certain proteins such as proteases, lactic acid, etc. Cells may be genetically modified, for example, using the methods commonly known in the art, such as by transfection, transformation or transduction, using recombinant expression vectors (see Examples and Sambrook et al., Molecular Cloning: A Laboratory Manual, 2nd ed., Cold Spring Harbor Press, Plainview, N.Y. (1989); Ausubel et al., Current Protocols in Molecular Biology (Supplement 47), John Wiley & Sons, New York (1999)). The vector may be integrated into chromosomal DNA or be carried as a resident plasmid by the genetically-modified cells. In some embodiments, retroviruses are used to genetically modify the cells. Additional genes that may be introduced into the cells are described in International PCT Publication No. WO99/37751.

In one embodiment, the cells can be genetically engineered to secrete a gene into the media which would exert a therapeutic effect, e.g., in the production of VEGF to stimulate neovascularization, or the production of angiotensin II to promote tissue repair after myocardial infarction. Since the constructs comprise eukaryotic cells, the gene product will be properly expressed and processed to form an active product. Preferably, the expression control elements used should allow for the regulated expression of the gene so that the product can be over-synthesized in culture. The transcriptional promoter chosen, generally, and promoter elements specifically, depend, in part, upon the type of tissue and cells cultured. The over-produced gene product will then be secreted by the engineered cell into the conditioned media. The cells may be genetically engineered to express protein(s) transiently or permanently. Similarly, expression of the protein may be inducible or noninducible.

The cells for generating the conditioned media can also be genetically engineered to “knock out” expression of factors that promote inflammation. Negative modulatory techniques for the reduction of target gene expression levels or target gene product activity levels are discussed below. “Negative modulation”, as used herein, refers to a reduction in the level and/or activity of target gene product relative to the level and/or activity of the target gene product in the absence of the modulatory treatment. The expression of a gene native to the cell can be reduced or knocked out using a number of techniques, for example, expression may be inhibited by inactivating the gene completely (commonly termed “knockout”) using standard homologous recombination techniques. Usually, an exon encoding an important region of the protein (or an exon 5′ to that region) is interrupted by a positive selectable marker (for example neo), preventing the production of normal mRNA from the target gene and resulting in inactivation of the gene. A gene may also be inactivated by creating a deletion or an inactivating insertion in part of a gene, or by deleting the entire gene. The sequences intervening the two regions can be deleted by using a construct with two regions of homology to the target gene that are far apart in the genome. Mombaerts et al., 1991, Proc. Nat. Acad. Sci. U.S.A. 88:3084-3087. Alternatively, a gene may also be inactivated by deletion of upstream or downstream expression elements.

In another embodiment, the cells may be engineered to express a target gene product which provides a chosen biological function, acts as a reporter of a chosen physiological condition, augments deficient or defective expression of a gene product, or provides an anti-viral, anti-bacterial, anti-microbial, or anti-cancer activity. In accordance with the present invention, the target gene product may be a peptide or protein, such as an enzyme, hormone, cytokine, antigen, or antibody, a regulatory protein, such as a transcription factor or DNA binding protein, a structural protein, such as a cell surface protein, or the target gene product may be a nucleic acid such as a ribosome or antisense molecule. The target gene products include, but are not limited to, gene products which enhance cell growth of the cultured cells. For example, the genetic modification may upregulate an endogenous protein, introduce a new protein or regulate ion concentration by expressing a heterologous ion channel or altering endogenous ion channel function. Examples include, but are not limited to engineered cells that express gene products which are delivered systemically (e.g., secreted gene products such as proteins including growth factors, hormones, Factor VIII, Factor IX, neurotransmitters, and enkaphalins).

In some embodiments, at least 0.5%, 1%, 5%, 10%, 20%, 50%, 80%, 90%, 95% or 98% of the cells in the population are genetically modified. In some embodiments, the cells are genetically modified to express a recombinant polypeptide, while in other embodiments the cells are genetically modified to express a double stranded RNA molecule, such as a hairpin RNA molecule, which inhibits the function of an endogenous gene. Preferred recombinant polypeptides include growth factors, chemokines, antibodies, cytokines, or receptors which bind to growth factors, chemokines, or cytokines. In another embodiment, the recombinant peptide is vascular endothelial growth factor (VEGF), hepatocyte growth factor (HGF), fibroblast growth factor (FGF), stromal cell-derived factor 1 (SDF-1), angiopoietin-1 or interleukin 8 (IL-8). In a preferred embodiment, the recombinant polypeptide is an angiogenic polypeptide, such as acidic fibroblast growth factor (aFGF), basic fibroblast growth factor (bFGF), vascular endothelial growth factor (VEGF-1), epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage CSF (M-CSF), angiopoetin-1 (Ang1) or nitric oxide synthetase (NOS); or effective fragments thereof In yet another embodiment, the recombinant polypeptide is CXCR-4, CXCR-5, VEGF-B, VEGF-C, VEGF-2, VEGF-3; or effective fragments thereof.

In another embodiment, the genetic modification promotes angiogenesis, vasculogenesis, or both. In another embodiment, the recombinant polypeptide is selected from the group consisting of leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors, FGF-acidic and basic fibroblast growth factor.

In another embodiment, the recombinant polypeptide expressed by the genetically modified cells promotes the proliferation, the differentiation or the ability of the cells in which it is produced, or in other cells present in the compositions, to localize to the ischemic tissue. In yet another embodiment, the genetic modification enhances the ability of the modified cells to interact with cells at the site of the ischemic tissue.

In another embodiment, the cells used to generate the conditioned media are genetically modified to express a suicide gene, or a conditionally-lethal gene. Such conditionally lethal genes allow the killing of cells upon treatment of the cells to particular agents. On one embodiment, the suicide gene is thymidine kinase or cytosine deaminase. Examples of suicide genes are described in U.S. Pat. No. 5,856,153.

In another embodiment, AC133+ cells or other cell types from which conditioned media is collected may be telomerized to increase their replicative capacity. Cells are telomerized by genetically altering them with a suitable vector so that they express the telomerase catalytic component (TERT) at an elevated level. Particularly suitable is the catalytic component of human telomerase (hTERT), provided in International Patent Publication WO 98/14592. For some applications, other TERT sequences can be used (mouse TERT is provided in WO 99/27113). Typically, the vector will comprise a TERT encoding region under control of a heterologous promoter that will promote transcription in the cell line. For example, sequences that can drive expression of the TERT coding region include viral LTRs, enhancers, and promoters (such as MPSV, SV40, MoLV, CMV, MSCV, HSV TK), eukaryotic promoters (such as β-actin, ubiquitin, EF1a, PGK) or combinations thereof (for example, the CMV enhancer combined with the β-actin promoter). Expression of a marker gene can be driven by the same promoter as the TERT gene, either as a separate expression cassette, as part of a polycistronic transcript (in which the coding regions of TERT and the marker gene are separated by an IRES sequence, allowing both individual proteins to be made from a single transcript driven by a single promoter), or as part of the same cassette (a fusion between the coding regions of both TERT and the marker gene, producing a protein that provides the functions of both TERT and the marker gene). Transfection and expression of telomerase in human cells is described in Bodnar et al., Science 279:349, 1998 and Jiang et al., Nat. Genet. 21:111, 1999.

Other methods of immortalizing cells are also contemplated, such as genetically altering the cells with DNA encoding the SV40 large T antigen (U.S. Pat. No. 5,869,243, International Patent Publication WO 97/32972), infecting with Epstein Bar Virus, introducing oncogenes such as myc and ras, introducing viral replication genes such as adenovirus E1a, and fusing cells having the desired phenotype with an immortalized cell line. Transfection with oncogenes or oncovirus products is usually less suitable when the cells are to be used for therapeutic purposes.

The “pre-conditioned” or nutrient cell culture medium may be any cell culture medium which adequately addresses the nutritional needs of the cells being cultured. Examples of cell media include, but are not limited to Dulbecco's Modified Eagle's Medium (DMEM), Ham's F12, RPMI 1640, Iscove's, McCoy's and other media formulations readily apparent to those skilled in the art, including those found in Methods For Preparation of Media, Supplements and Substrate For Serum-Free Animal Cell Culture Alan R. Liss, New York (1984) and Cell & Tissue Culture: Laboratory Procedures, John Wiley & Sons Ltd., Chichester, England 1996, both of which are incorporated by reference herein in their entirety. In one preferred embodiment, the AC133+ cells or other cell types are cultured in serum free media. Serum free media is described, for example, in U.S. Pat. Nos. 5,766,951, 5,945,337 and 6,733,746.

The nutrient medium may be supplemented, with any components necessary to support the desired cell or tissue culture. Additionally, the concentrations of the ingredients are well known to one of ordinary skill in the art. The ingredients include amino-acids (both D and/or L-amino acids) such as glutamine, alanine, arginine, asparagine, cysteine, glutamic acid, glycine, histidine, isoleucine, leucine, lysine, methionine, phenylalanine, proline, serine, threonine, tryptophan, tyrosine, and valine and their derivatives; acid soluble subgroups such as thiamine, ascorbic acid, ferric compounds, ferrous compounds, purines, glutathione and monobasic sodium phosphates. Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers. Other ingredients often used in media formulations include fat soluble vitamins (including A, D, E and K) steroids and their derivatives, cholesterol, fatty acids and lipids Tween 80, 2-mercaptoethanol pyrimidines as well as a variety of supplements including serum (fetal, horse, calf, etc.), proteins (insulin, transferrin, growth factors, hormones, etc.) antibiotics (gentamicin, penicillin, streptomycin, amphotericin B, etc.) whole egg ultra filtrate, and attachment factors (fibronectins, vitronectins, collagens, laminins, tenascins, etc.). Erythropoietin may be added to the nutrient medium to increase the potency of the conditioned media for inducing neovascularization when administered to a subject.

In one embodiment, the nutrient medium is supplemented with albumin. Albumin is preferably supplied in the form of human serum albumin (HSA) in an effective amount for the growth of cells. HSA provides a source of protein in the media. Moreover, HSA acts as a substrate for proteases which might otherwise digest cell membrane proteins. Albumin is thought to act as a carrier for trace elements and essential fatty acids. HSA is greatly advantageous over protein derived from animals such as bovine serum albumin (BSA) due to the reduced immunogenic potential of HSA once the resulting conditioned medium is administered to a subject. HSA may be recombinantly produced in such hosts as bacteria or yeast, or in vegetable cells such as potato and tomato. Alternatively, in a less preferred embodiment, HSA may be derived from pooled human plasma fractions. Preferably, the HSA used in the present formulations is free of pyrogens and viruses, and is approved by regulatory agencies for infusion into human patients. The HSA may be deionized using resin beads prior to use. The concentration of human serum albumin in the preconditioned media may be 1-8 mg/ml, preferably 3-5 mg/ml, and most preferably about 4 mg/ml.

In another embodiment, in addition to or in lieu of albumin, a soluble carrier/essential fatty acid complex and a soluble carrier cholesterol complex which can effectively deliver the fatty acid and cholesterol to the cells is added to the preconditioned medium. An example of such a complex is a cyclodextrin/linoleic acid, cholesterol and oleic acid complex. This is advantageous as it would allow for the replacement of the poorly characterized albumin with a well defined molecule, especially when the conditioned media is to be administered to a subject. The use of cyclodextrin removes the need for the addition of human/animal serum albumin, thereby eliminating any trace undesired materials which the albumin would introduce into the media. The use of cyclodextrin simplifies the addition of specific lipophilic nutrients to a serum-free culture.

In one embodiment, serum, such as bovine serum, which is a complex solution of albumins, globulins, growth promoters and growth inhibitors, may be added to the nutrient media. The serum should be pathogen free and carefully screened for mycoplasma bacterial, fungal, and viral contamination. Also, the serum should generally be obtained from the United States and not obtained from countries where indigenous livestock carry transmittable agents. Hormone addition into the medium may or may not be desired.

In another embodiment, the nutrient medium is supplemented with a source of iron in an effective amount and in a form that can be utilized by the AC133+ cells, or by the other cell types. The iron can be supplied by transferrin in an effective amount. The transferrin may be derived from animal sera or recombinantly synthesized. It is understood that when transferrin is derived from an animal source, it is purified to remove other animal proteins, and thus is usually at least 99% pure. The transferrin concentration is usually between 80 and 500 μg/ml, preferably between 120 and 500 μg/ml, more preferably between 130 and 500 μg/ml, even more preferably between 275 and 400 μg/ml and most preferably about 300 μg/ml. Alternatively, an iron salt, preferably a water soluble iron salt, such as iron chloride (e.g. FeCl₃.6H₂O) dissolved in an aqueous solution such as an organic acid solution (e.g. citric acid) can be used to supply the iron. One mole of iron chloride is usually used for every mole of citric acid. The concentration of iron chloride is from 0.0008 to 8 μg/ml, preferably from 0.08 to 0.8 μg/ml.

In one embodiment, the cells are cultured under hypoxic conditions to increase the release into the medium of components that support neovascularization. In one embodiment, the hypoxic conditions comprise growing the cell population at an oxygen concentration of 4%-12%, or more preferably at an oxygen concentration of 5%-7%.

General techniques in cell culture and media collection are outlined in Large Scale Mammalian Cell Culture (Hu et al., Curr. Opin. Biotechnol. 8:148, 1997); Serum-free Media (K. Kitano, Biotechnology 17:73, 1991); Large Scale Mammalian Cell Culture (Curr. Opin. Biotechnol. 2:375, 1991); and Suspension Culture of Mammalian Cells (Birch et al., Bioprocess Technol. 19:251, 1990).

The selected culture medium is then combined with the cells used for conditioning in an environment that allows the cells to release into the medium the components that support neovascularization. The cells may be cultured in any manner known in the art including in monolayer, beads or in three-dimensions and by any means (i.e., culture dish, roller bottle, a continuous flow system, etc.). Methods of cell and tissue culturing are well known in the art, and are described, for example, in Cell & Tissue Culture: Laboratory Procedures, John Wiley & Sons Ltd., Chichester, England 1996; Freshney, Culture of Animal Cells: A Manual of Basic Techniques, 2d Ed., A. R. Liss, Inc., New York, 1987, Ch. 11 and 12, pp. 137-168.

In one embodiment, culture dishes are coated with extracellular matrix components to promote cell adherence and/or growth. Particularly suitable are extracellular matrix components, such as those derived from basement membrane or that may form part of adhesion molecule receptor-ligand couplings. A commercial preparation is available from Becton Dickenson under the name Matrigel®, and can be obtained in a Growth Factor Reduced formulation. Other extracellular matrix components and component mixtures are suitable as an alternative. Depending on the cell type being proliferated, this may include laminin, fibronectin, proteoglycan, entactin, heparan sulfate, and the like, alone or in various combinations. Laminins are major components of all basal laminae in vertebrates, which interact with integrin heterodimers such as α6β1 and α6β4 (specific for laminins) and other heterodimers (that cross-reach with other matrices).

In some embodiments, the cells are grown in synthetic matrices composed of biodegradable, biocompatible copolymers of polyesters and amino acids, that have been designed as scaffolding for cell growth (U.S. Pat. Nos. 5,654,381; 5,709,854). Non-biodegradable scaffolds are likewise capable of supporting cell growth. Three-dimensional cell culture systems have also been designed which are composed of a stromal matrix which supports the growth of cells from any desired tissue into an adult tissue (Naughton et al, U.S. Pat. Nos. 4,721,096 and 5,032,508).

Selection of culture apparatus for conditioning medium can be made based on the scale and purpose of medium collection. In initial studies and for screening purposes, it is often convenient to produce cultured medium in standard culture flasks or multi-well plates. Large scale, automated, or GMP compliant production can involve the use of specialized devices. Continuous cell culture systems are reviewed by J. Furey (Genetic Eng. News 20:10, May 15, 2000). Perfusion culture involves removal of medium from the culture chamber, and replenishment with fresh medium. In the spin basket system, a basket-like device is attached to a drive shaft and covered by a porous screen through which medium can be exchanged. In the external filter perfusion system, a culture is circulated from a vessel, through a hollow-fiber filter module, and back to the vessel, with a pump attached to the loop to provide the circulation. A particular perfusion system, the ATF System (available commercially from Refine Technology, Edison N.J.) consists of a diaphragm pump on one end of a hollow-fiber housing, the other end of which is connected to a bioreactor. Alternating tangential flow through the fibers generates low shear laminar flow, which provides high flow rates, scalability, and adaptability to different bioreactors.

Large-scale culture systems are also available from Aastrom Sciences Inc., Ann Arbor Mich. The Aastrom Replicell™ System provides for expansion from small starting cell populations (Koller et al., Bone Marrow Transpl. 21:653, 1009; Koller et al., Blood 86:1784,1995). Cellstasis® culture technology is marketed by Genespan Corp., Bothell Wash. Cells reside in extracapilliary spaces, and hollow fibers bring fresh media and oxygen into the culture environment (R. Lewis, Genetic Eng. News18(9), May 1, 1998). Any other suitable device can be used with this invention. U.S. Pat. No. 4,501,815 describes a device for culturing differentiated cells. U.S. Pat. No. 4,296,205 describes cell culture and continuous dialysis flasks and their use. U.S. Pat. No. 5,994,129 describes a portable cassette for use in maintaining biological cells. U.S. Pat. No. 5,362,642 describes a containment system for storing, reconstituting, dispensing, and harvesting cell culture media. U.S. Pat. No. 6,022,742 describes a culture device and method.

The cells can be cultured by any means known in the art. Preferably, the cells are cultured in an environment which enables aseptic processing and handling. Conventional means of cell and tissue culture have been limited by the need for human supervision and control of the media. This limits the amount of cells and tissue that can be cultured at a single time and consequently the volume of conditioned cell media that can be obtained at a single time. For this reason, it is preferred that the media be conditioned in a manner allowing for large scale growth (yielding large scale conditioned media) using, for example, an apparatus for aseptic large scale culturing like that described in co-owned U.S. Pat. No. 5,763,267 (the '267 patent) which is incorporated by reference herein in its entirety. See also, U.S. Pat. No. 5,843,766 (also incorporated herein in its entirety) which describes an apparatus for aseptic growth of three-dimensional tissue cultures. Using the aseptic closed system described in the '267 patent, preconditioned culture media is transported from a fluid reservoir to an inlet manifold and evenly distributed to the cultures in a continuous flow system. When appropriate, (i.e., once the media is conditioned so that the extracellular proteins such as growth factors have reached desirable levels in the media) it is pumped out of the system and processed for use.

In some embodiments, the cells can be inactivated (i.e., rendered incapable of substantial replication) by radiation (e.g., about 4,000 rads), treatment with a chemical inactivator like mitomycin C, or by any other effective method, prior to, during, or after culturing in preconditioned media.

The cells are cultured in the medium for sufficient time to allow adequate concentration of released factors (or consumption of media components) to produce a medium that supports neovascularization. In one embodiment, the medium is conditioned by culturing for 24 h at 37° C. However, the culturing period can be adjusted upwards or downwards, determining empirically (or by assaying for the concentration of essential factors) what constitutes an adequate period. After collecting a batch of conditioned medium, the cells can be used to condition a further batch of medium over a further culture period, for as many cycles as desired as long as the cells retain their ability to condition the medium in an adequate fashion.

Following removal of the cell conditioned medium, it may be necessary to further process the resulting supernatant. Such processing may include, but is not limited to, concentration by a water flux filtration device or by defiltration using the methods described in Cell & Tissue Culture: Laboratory Procedures, John Wiley & Sons Ltd., Chichester, England 1996, pp 29 D:0.1-29D:0.4.

Additionally, the conditioned medium may be further processed for product isolation and purification to remove unwanted proteases, for example. The methods used for product isolation and purification so that optimal biological activity is maintained will be readily apparent to one of ordinary skill in the art. Also, the medium may be further processed to concentrate or reduce one or more factors or components contained within the medium, for example, enrichment of a growth factor using immunoaffinity chromatography or, conversely, removal of a less desirable component, for any given application as described herein. For example, it may be desirable to purify a growth factor, regulatory factor, peptide hormone, antibody, etc.

Methods that may be used to concentrate a component or to exclude a component from the conditioned medium include, but are not limited to, gel chromatography (using matrices such as sephadex) ion exchange, metal chelate affinity chromatography with an insoluble matrix such as cross-linked agarose, HPLC purification and hydrophobic interaction chromatography of the conditioned media. Such techniques are described in greater detail in Cell & Tissue Culture: Laboratory Procedures, John Wiley & Sons Ltd., Chichester, England 1996. Depending upon the desired application of the conditioned medium, and/or products derived thereof, appropriate measures must be taken to maintain sterility. Alternatively, sterilization may be necessary and can be accomplished by methods known to one of ordinary skill in the art, such as, for example, heat and/or filter sterilization taking care to preserve the desired biological activity. It may be preferable to remove cellular debris or other particular matter as well as proteases or lactic acid.

Therapeutic products contained in the conditioned media which may be concentrated include, but are not limited to, enzymes, hormones, cytokines, antigens, antibodies, clotting factors, and regulatory proteins. Therapeutic proteins include, but are not limited to, inflammatory mediators, angiogenic factors, Factor VIII, Factor IX, erythropoietin, alpha-1 antitrypsin, calcitonin, glucocerebrosidase, human growth hormone and derivatives, low density lipoprotein (LDL), Erythropoietin (EPO), and apolipoprotein E, IL-2 receptor and its antagonists, insulin, globin, immunoglobulins, catalytic antibodies, the interleukins, insulin-like growth factors, superoxide dismutase, immune responder modifiers, BMPs (bone morphogenic proteins) parathyroid hormone and interferon, nerve growth factors, tissue plasminogen activators, and colony stimulating factors.

One aspect of the invention provides compositions comprising conditioned media or concentrated or purified components from the conditioned media. The compositions may be liquid, solid, lyophilized, cryopreserved, semisolid or gelatinous compositions. In one embodiment, the composition is supplemented with such additives as antibiotics, antivirals, antifungals, steroids, analgesics, antitumor drugs, investigational drugs or any compounds which would result in a complimentary or synergistic combination with the neovascularization factors in the conditioned media.

In another embodiment, the conditioned medium may be formulated with a pharmaceutically acceptable carrier as a vehicle for internal administration. The conditioned media of the invention can be formulated into injectable preparations. Alternatively, products derived from the conditioned media can be formulated. For example, biologically active substances, such as proteins and drugs, can be incorporated in the compositions of the present invention for release or controlled release of these active substances after injection of the composition into the subject.

In one preferred embodiment, the compositions comprising conditioned media or concentrated or purified components from the conditioned media are pharmaceutical compositions. Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, by aerosol, oral, topical or intravenous route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular, otic delivery or implantation.

Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In the injectable embodiment, an aqueous suspension is used and the formulation of the aqueous suspension will typically have a physiological pH (i.e., about pH 6.8 to 7.5). In one embodiment, a local anesthetic, such as lidocaine, (usually at a concentration of about 0.3% by weight) is usually added to reduce local pain upon injection. The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, preferably succinylated collagen, can also act as lubricants. Such lubricants are generally used to improve the injectability, intrudability and dispersion of the injected biomaterial at the site of injection and to decrease the amount of spiking by modifying the viscosity of the compositions.

In addition to the formulations described previously, the compounds may also be formulated as a depot preparation. Such long acting formulations may be administered by implantation (for example subcutaneously or intramuscularly) or by intramuscular or intracardiac injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art.

In one embodiment, the composition further comprises a biologically active agent, such as an antiinflammatory agent. Antiinflammatory agents include, but are not limited to, any known non-steroidal antiinflammatory agent, and any known steroidal antiinflammatory agent. Antiinflammatory agents include, but are not limited to, any known nonsteroidal antiinflammatory agent such as, salicylic acid derivatives (aspirin), para-aminophenol derivatives(acetaminophen), indole and indene acetic acids (indomethacin), heteroaryl acetic acids (ketorolac), arylpropionic acids (ibuprofen), anthranilic acids (mefenamic acid), enolic acids (oxicams) and alkanones (nabumetone) and any known steroidal antiinflammatory agent which include corticosteriods and biologically active synthetic analogs with respect to their relative glucocorticoid (metabolic) and mineralocorticoid (electrolyte-regulating) activities. Additionally, other drugs used in the therapy of inflammation or antiinflammatory agents including, but are not limited to, the autocoid antagonists such as all histamine and bradykinin receptor antagonists, leukotriene and prostaglandin receptor antagonists, and platelet activating factor receptor antagonists.

In another embodiment, the composition further comprises an antimicrobial agents, such as antibiotics (e.g. antibacterial), antiviral agents, antifungal agents, and anti-protozoan agents. Non-limiting examples of antimicrobial agents are sulfonamides, cephalosporins, trimethoprim-sulfamethoxazole, quinolones and penicillins.

In another embodiment, the composition further comprises an antineoplastic agent, such as but not limited to, those which are suitable for treating tumors that may be present on or within an organ (e.g., myxoma, lipoma, papillary fibroelastoma, rhabdomyoma, fibroma, hemangioma, teratoma, mesothelioma of the AV node, sarcomas, lymphoma, and tumors that metastasize to the target organ) including cancer chemotherapeutic agents, a variety of which are well known in the art.

In another embodiment, the composition further comprises an angiogenic factor (e.g., to promote organ repair or for development of a biobypass to avoid a thrombosis) including but not limited to, basic fibroblast growth factor, acidic fibroblast growth factor, vascular endothelial growth factor, angiogenin, transforming growth factor α and β, tumor necrosis factor, angiopoietin, platelet-derived growth factor, placental growth factor, hepatocyte growth factor and proliferin.

In another embodiment, the composition further comprises a thrombolytic agent including, but not limited to, urokinase plasminogen activator, urokinase, streptokinase, inhibitors of α2-plasmin inhibitor, and inhibitors of plasminogen activator inhibitor-1, angiotensin converting enzyme (ACE) inhibitors, spironolactone, tissue plasminogen activator (tPA), an inhibitor of interleukin 1β converting enzyme, anti-thrombin III, and the like.

In another embodiment, the composition further comprises an antihypertensive agent including, but not limited to, diuretics, including thiazides such as hydroclorothiazide, furosemide, spironolactone, triamterene, and amiloride; antiadrenergic agents, including clonidine, guanabenz, guanfacine, methyldopa, trimethaphan, reserpine, guanethidine, guanadrel, phentolamine, prazosin, phenoxybenzamine, terazosin, doxazosin, propanolol, methoprolol, nadolol, atenolol, timolol, betaxolol, carteolol, pindolol, acebutolol, labetalol; vasodilators, including hydralizine, minoxidil, diazoxide, nitroprusside; and angiotensin converting enzyme inhibitors, including captopril, benazepril, enalapril, enalaprilat, fosinopril, lisinopril, quinapril, ramipril; angiotensin receptor antagonists, such as losartan; and calcium channel antagonists, including nifedine, amlodipine, felodipine XL, isadipine, nicardipine, benzothiazepines (e.g., diltiazem), and phenylalkylamines (e.g. verapamil).

In another embodiment, the composition further comprises an anti-coagulant including, but not limited to, heparin; warfarin; hirudin; tick anti-coagulant peptide; low molecular weight heparins such as enoxaparin, dalteparin, and ardeparin; ticlopidine; danaparoid; argatroban; abciximab; and tirofiban.

In another embodiment, the composition further comprises an antiarrhythmic agent including but not limited to, sodium channel blockers (e.g., lidocaine, procainamide, encainide, flecanide, and the like), beta adrenergic blockers (e.g., propranolol), prolongers of the action potential duration (e.g., amiodarone), and calcium channel blockers (e.g., verpamil, diltiazem, nickel chloride, and the like). Delivery of cardiac depressants (e.g., lidocaine), cardiac stimulants (e.g., isoproterenol, dopamine, norepinephrine, etc.), and combinations of multiple cardiac agents (e.g., digoxin/quinidine to treat atrial fibrillation) is also of interest.

In another embodiment, the composition further comprises a therapeutically effective amount of platelet microparticles. Platelet microparticles are described in U.S. Pat. No. 5,185,160 and in Nomura S, and Fukuhara S. Methods Mol Biol. 2004;272:269-77 and in Kim et al. Br J Haematol. February 2004;124(3):376-84. In one embodiment, the microparticles are autologous to the subject.

In another embodiment, the composition further comprises an siRNA, hairpin RNA, or other nucleic acid designed to knock down expression of a gene or set of genes. Hairpin and siRNAs are described, for example, in U.S. Patent Pub Nos. 2004/0053876, 2004/0229266, 2004/0053289 and 2004/0058886.

In one embodiment, the composition further comprises a virus expressing a therapeutic gene, such as a gene which promotes angiogenesis or neovascularization Therapeutic polypeptides include PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, erythropoietin, Osteopontin, Pleiotropin, Activin and Endothelin-1. In one embodiment, the polypeptide is VEGF or TGFβ1 or both.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by a nucleic acid encoding a CKI polypeptide, rendering the retrovirus replication defective. The replication defective retrovirus is then packaged into virions which can be used to infect a target cell through the use of a helper virus by standard techniques.

Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al. (1985) Science 230:1395-1; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

The composition may comprise conditioned media components from two or more cell populations which have been cultured together or separately. In one embodiment, the therapeutic compositions are derived from conditioned cell culture medium obtained by co-culturing (i) the first cell population; and (b) the second cell population. In another embodiment, the conditioned cell culture medium is obtained by separately culturing (i) the first cell population; and (b) the second cell population. For example, in one preferred embodiment, AC133+ cells are cultured together with mesenchymal stem cells such that the resulting conditioned media contains factors from both cell populations. In a related embodiment, AC133+ cells and mesenchymal stem cells are cultured separately, such that two sets of conditioned medium are isolated. Components from each conditioned medium may then be combined to form a composition, which may also comprise additional factors. In another preferred embodiment, CD34+ cells, EPCs or MAPCs are cultured with mesenchymal stem cells such that the resulting conditioned media contains factors from both cell types.

Another aspect of the invention provides compositions comprising (i) conditioned media or (ii) components from conditioned media, which are formulated with polymerizable or cross-linking hydrogels, such as is described in U.S. Pat. Nos. 5,709,854; 5,516,532; or 5,654,381; or as described in WO 98/52543, each of which is incorporated herein by reference in its entirety. Examples of materials which can be used to form a hydrogel include modified alginates. Alginate is a carbohydrate polymer isolated from seaweed, which can be cross-linked to form a hydrogel by exposure to a divalent cation such as calcium, as described, for example in WO94/125080, the disclosure of which is incorporated herein by reference. Alginate is ionically cross-linked in the presence of divalent cations, in water, at room temperature, to form a hydrogel matrix. As used herein, the term “modified alginates” refers to chemically modified alginates with modified hydrogel properties. In another embodiment, the composition is formulated as gelatin hydrogel microspheres. For example, Hosaka et al. (2004) Circulation.; 110(21):3322-8 and Linn et al. (2003) Cell Transplant; 12(7): 769-78 describe chitosan-based, PLGA-based or acidic gelatin-based hydrogel microspheres suitable for administration of the therapeutic compositions described herein. These microspheres are suitable for intravenous and intra-arterial administration.

Additionally, polysaccharides which gel by exposure to monovalent cations, including bacterial polysaccharides, such as gellan gum, and plant polysaccharides, such as carrageenans, may be cross-linked to form a hydrogel using methods analogous to those available for the cross-linking of alginates described above. Modified hyaluronic acid derivatives are particularly useful. As used herein, the term “hyaluronic acids” refers to natural and chemically modified hyaluronic acids. Modified hyaluronic acids may be designed and synthesized with preselected chemical modifications to adjust the rate and degree of cross-linking and biodegradation. Covalently cross-linkable hydrogel precursors also are useful. For example, a water soluble polyamine, such as chitosan, can be cross-linked with a water soluble diisothiocyanate, such as polyethylene glycol diisothiocyanate. Alternatively, polymers may be utilized which include substituents which are cross-linked by a radical reaction upon contact with a radical initiator. For example, polymers including ethylenically unsaturated groups which can be photochemically cross-linked which may be utilized, as disclosed in WO 93/17669, the disclosure of which is incorporated herein by reference. In this embodiment, water soluble macromers that include at least one water soluble region, a biodegradable region, and at least two free radical-polymerizable regions, are provided. Examples of these macromers are PEG-oligolactyl-acrylates, wherein the acrylate groups are polymerized using radical initiating systems, such as an eosin dye, or by brief exposure to ultraviolet or visible light. Additionally, water soluble polymers which include cinnamoyl groups which may be photochemically cross-linked may be utilized, as disclosed in Matsuda et al., ASAID Trans., 38:154-157 (1992).

The preferred polymerizable groups are acrylates, diacrylates, oligoacrylates, dimethacrylates, oligomethacrylates, and other biologically acceptable photopolymerizable groups. Acrylates are the most preferred active species polymerizable group. Naturally occurring and synthetic polymers may be modified using chemical reactions available in the art and described, for example, in March, “Advanced Organic Chemistry”, 4^{th} Edition, 1992, Wiley-Interscience Publication, New York. Polymerization is preferably initiated using photoinitiators. Useful photoinitiators are those which can be used to initiate polymerization of the macromers without cytotoxicity and within a short time frame, minutes at most and most preferably seconds. Numerous dyes can be used for photopolymerization. Suitable dyes are well known to those of skill in the art. Preferred dyes include erythrosin, phioxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable cocatalysts include amines such as N-methyl diethanolamine, N,N-dimethyl benzylamine, triethanol amine, trithylamine, dibenzyl amine, N-benzylethanolamine, -isopropyl benzylamine. Triethanolamine is a preferred cocatalyst.

In yet another embodiment, the composition is formulated with a material capable of polymerizing or gelling after implantation into a mammal. The polymerizing or gelling after implantation may be initiated by thermal, enzymatic or chemical catalysts, pH or ionic strength changes or photo-initiation procedures.

Another aspect of the invention provides a implantable devices useful for administering the conditioned media compositions described herein to a subject, such as to a mammal. U.S. Pat. No. 6,455,074 describes polymer-based controlled release devices and methods of fabricating them. Biocompatible, non-biodegradable polymers suitable for a sustained release device include non-biodegradable polymers selected from the group consisting of polyacrylates, polymers of ethylene-vinyl acetates and acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. The implantable device may comprise a matrix from which the composition is released. Matrices may comprise a biopolymer selected from the group consisting of collagen, gelatin, hyaluronic acid or chemically derived modifications of hyaluronic acid, chitin, chitosan or chitosan derivatives, fibrin, dextran, agarose, or calcium alginate. In one preferred embodiment, the matrix is a biodegradable matrix. In one embodiment, the matrix comprises a synthetic polymeric material selected from the group consisting of polylactic acid, polyglycolic acid or copolymers or combinations of the two, polyurethanes, polycarbonates, polycaprolactones, polyacrylates, polyvinyl alcohols, polyethylene glycols, or polyethyleneimines. In another embodiment, the matrix comprises a tissue particle selected from the group consisting of bone or demineralized bone, cartilage, tendon, ligament, fascia, intestinal mucosa or other connective tissues, or chemically modified derivatives thereof.

In one embodiment, isolated polypeptides and other macromolecules from the conditioned media are formulated for admistration into a subject using a derivatized hyaluronic acid (Hyaff-11) scaffold as a delivery vehicle. Such delivery vehicle has been shown to be effective in the administration of the growth factor BMP-2 (see Kim et al. J Biomed Mater Res 59: 573-584, 2002).

In addition, the sustained release devices of the instant invention can also contain other excipients, such as stabilizers, bulking agents or aggregation-stabilizing agents. Stabilizers are added to maintain the potency of the biologically active agent during device fabrication, storage and over the duration of the release of media components. Suitable stabilizers include, for example, carbohydrates, amino acids, fatty acids and surfactants which are known to those skilled in the art.

In one embodiment, submicron particles of a conditioned media or of compositions comprising conditioned media components are prepared by atomizing the media of composition and at least one solvent to produce droplets, freezing the droplets to produce frozen droplets, lyophilizing the frozen droplets to obtain microstructures capable of being further fragmented into submicron particles by techniques such as probe sonication as described in U.S. Pat. No. 6,428,815. The submicron particles can be incorporated into sustained release devices.

In another embodiment, the conditioned media or its components may be formulated into a drug delivery vehicle that may be visualized noninvasively with MRI as described in Faranesh et al. (2004) Magn Reson Med. 51(6):1265-71. In a specific embodiment, the biodegradable polymer poly(DL-lactic-co-glycolic acid) (PLGA) is used to fabricate microspheres containing the conditioned media components and the MRI contrast agent gadolinium diethylenetriamine pentaacetic acid (Gd-DTPA). Such microspheres can be visualized non-invasively under MRI and allow the release of polypeptide factors over a period of approximately 6 weeks.

In another embodiment, the invention provides stents comprising conditioned media or components thereof, which may be coated onto its surface. A stent is a generally longitudinal tubular device formed of biocompatible material, preferably a metallic or plastic material. Stents are useful in the treatment of stenosis, strictures or aneurysms in body vessels, such as blood vessels. It is well-known to employ a stent for the treatment of diseases of various body vessels. The device is implanted either as a “permanent stent” within the vessel to reinforce collapsing, partially occluded, weakened or abnormally dilated sections of the vessel or as a “temporary stent” for providing therapeutic treatment to the diseased vessel. Stents are typically employed after angioplasty of a blood vessel to prevent restenosis of the diseased vessel. Preferred embodiments include coronary stents, polymer coated stents and drug-eluting stents. Drug-eluting stents which may be coated with conditioned media components of the present invention are well-known in the art (reviewed in Nelken et al. Surg Clin North Am. 2004;84(5):1203-36; Panescu et al. IEEE Eng Med Biol Mag. 2004; 23(2):21-3). Drug eluting stents are also described in U.S. Patent Pub. Nos. 2004/0215315, 2004/0204750, 2004/0093064, 2004/002367, 2003/0216803, 2003/0216803 and in U.S. Pat. Nos. 5,591,227 and 5,697,967.

The compositions described herein may further comprise at least one type of cell. Preferred cell types are those which induce vascularization when administered into a subject, and include AC133+ cells; (ii) endothelial precursor cells; (iii) MAPCs; (iv) mesenchymal stem cells; (v) AC133-CD34-CD73-cells; (vi) CD34+ cells.

IV. Methods of Inducing Neovascularization

Some aspects of the invention provide methods of treating or preventing a disorder. Some aspects provide methods of treating disorders which are associated with ischemia, vascular occlusion, reduced blood circulation or reduced vascularization. One aspect of the invention provides a method of inducing neovascularization in a subject in need thereof, the method comprising administering to the subject a therapeutically effective amount of any of the conditioned media compositions described herein. In one preferred embodiment of the methods for inducing neovascularization, the subject is a human. In another embodiment, the subject is an adult, a newborn, an embryo or a fetus.

One specific aspect provides a method of inducing neovascularization in a subject in need thereof, the method comprising administering to the subject a therapeutically effective amount of a composition comprising conditioned cell culture medium from a first population of cells comprising (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In one preferred embodiment, the first population of cells is derived from umbilical cord blood, which may be from a single umbilical cord or from a plurality of umbilical cords. In one embodiment, at least 5%, 10%, 20%, 30%, 40%, 50%, 60%, 70%, 80%, 90%, 95% or greater of the cells in the first population are (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In a preferred embodiment, the CD133+ cells are CD133+CD34+KDR-CXCR4-cells. In one embodiment, at least one cell in the first population of cells is genetically modified, such as genetically modified to express a transgene. Transgenes include cytokine, chemokines, growth factors, antibodies, adhesion factors, extracellular matrix proteins or integrins. In a specific embodiment, the transgene is selected from the group consisting of PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof. In one embodiment, the transgene is VEGF or TGFβ1 or both.

In one embodiments, the composition also includes a conditioned media component from a second population of cells that is different from the second population of cells, whereas in a related embodiment a second composition is administered to the subject which comprises a conditioned media component from a second population of cells that is different from the second population of cells. In one embodiment, the second population of cells comprises (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In one preferred embodiment, the second population of cells is derived from umbilical cord blood, which may be from a single umbilical cord or from a plurality of umbilical cord. In one embodiment, at least 5%, 10%, 20%, 30%, 40%, 50%, 60%, 70%, 80%, 90%, 95% or greater of the cells in the second population are (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. In a preferred embodiment, the CD133+ cells are CD133+CD34+KDR-CXCR4-cells. In one embodiment, at least one cell in the second population of cells is genetically modified, such as genetically modified to express a transgene. A transgene might include a cytokine, chemokine, growth factor, antibody, adhesion factor, extracellular matrix protein or an integrin. In a specific embodiment, the transgene is selected from the group consisting of PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof. In one embodiment, the transgene is VEGF or TGFβ1 or both.

In one embodiment, the therapeutic compositions are derived from conditioned cell culture medium obtained by co-culturing (i) the first cell population; and (b) the second cell population. In another embodiment, the conditioned cell culture medium is obtained by separately culturing (i) the first cell population; and (b) the second cell population. In one preferred embodiment, the first population of cells, the second population of cells, or both, are cultured under hypoxic conditions. In one embodiment, the hypoxic conditions comprise growing the cell population at an oxygen concentration of 4%-12%, or more preferably at an oxygen concentration of 5%-7%.

There are numerous conditions that cause the necessity of a subject to be in need of neovascularization. For example, the subject may have a wound that requires healing. The wound may be an acute wound, such as those caused by burns and contact with hard and/or sharp objects. For example, patients recovering from surgery, such as cardiovascular surgery, cardiovascular angioplasty, carotid angioplasty, and coronary angioplasty all require neovascularization. The wound may also be a chronic wound. Some examples of chronic wounds include ulcers, such as vascular ulcers and diabetic ulcers. The compositions and devices of the present invention may be used in increasing cardiac or peripheral (i.e. limb) vascularization. Therefore, the methods of the present invention are especially desirable in treating cardiac and peripheral ischemia. Patients suffering from other conditions also require neovascularization. Such conditions include sickle cell anemia and thalassemia.

In one embodiment of the methods described herein for the inducement of neovascularization in a subject, the subject is afflicted with ischemia. The present methods are not limited to ischemia in any particular tissue, but are applicable to any type of ischemia. For example, in one embodiment, the subject is afflicted with ischemia in multiple tissues. In such embodiment, a systemic infusion of cells to the subject may be performed, or alternatively or in combination, one or more localized infusions near the ischemic tissue may be performed. In one embodiment of the methods described herein, the ischemic myocardium comprises an area of viable myocardium. In a related embodiment, the ischemia is selected from the group consisting of cerebrovascular ischeria, renal ischemia, pulmonary ischemia, limb ischemia, ischemic cardiomyopathy and myocardial ischemia.

In one preferred embodiment of the methods described herein, the subject afflicted with an ischemic tissue is in need of treatment for chronic myocardial ischemia. In one specific embodiment of the methods described herein for the treatment of ischemia, the ischemia is selected from the group consisting of cardiac, peripheral vascular, cerebral and renal ischemia. In a specific embodiment, the subject is afflicted with at least one ischemic condition selected from the group consisting of myocardial infarction, angina pectoris, any cardiac surgical interventions, circulatory insufficiency in extremities, ischemia-reperfusion injury, stroke, trauma and peripheral vascular disease (PVD).

In one embodiment, the compositions are formulated for intracoronary, intravenous, intradermal, intraarterial, intramuscular, intracardiac, intraorbital, intraspinal or subcutaneous administration to the subject. In one embodiment, the composition is administered as a solid, lyophilate, powder, gel, film or hydrogel.

In one embodiment, the composition is administered via an implantable device, which may, for example, be implanted at or near a site of ischemia. In one embodiment, the implantable device is a sustained release device. In one embodiment, the implantable device is a stent. Preferred embodiments include coronary stents, polymer coated stents and drug-eluting stents.

In another embodiment, the composition is administered via a gelatin hydrogel microsphere. For example, Hosaka et al. Circulation. 2004;110(21):3322-8 and Linn et al. Cell Transplant. 2003; 12(7): 769-78 describe chitosan-based, PLGA-based or acidic gelatin-based hydrogel microspheres suitable for administration of the therapeutic compositions described herein. These microspheres are suitable for intravenous and intra-arterial administration.

The implantable device may comprise a matrix from which the compositions is released. Matrices may comprise a biopolymer selected from the group consisting of collagen, gelatin, hyaluronic acid or chemically derived modifications of hyaluronic acid, chitin, chitosan or chitosan derivatives, fibrin, dextran, agarose, or calcium alginate. In one preferred embodiment, the matrix is a biodegradable matrix. In one embodiment, the matrix comprises a synthetic polymeric material selected from the group consisting of polylactic acid, polyglycolic acid or copolymers or combinations of the two, polyurethanes, polycarbonates, polycaprolactones, polyacrylates, polyvinyl alcohols, polyethylene glycols, and polyethyleneimines. In another embodiment, the matrix comprises a tissue particle selected from the group consisting of bone or demrineralized bone, cartilage, tendon, ligament, fascia, intestinal mucosa or other connective tissues, or chemically modified derivatives thereof.

In yet another embodiment, the composition comprises a material capable of polymerizing or gelling after implantation into said mammal. The polymerizing or gelling after implantation may be initiated by thermal, enzymatic or chemical catalysts, pH or ionic strength changes or photo-initiation procedures.

One embodiment of the methods described herein for inducing neovascularization in a subject further comprises administering to the subject a therapeutic amount of at least one type of cell, preferably a therapeutic cell which induces neovascularization in the subject. The cells that are administered to the subject may be autologous, allogenic, or HLA compatible with the subject. In one embodiment, the therapeutic cells are selected from the group consisting of (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof. The therapeutic cells may be different or the same type of cells as the cells in the first cell population.

The number of cells administered to an individual afflicted with an ischemic tissue will vary according to the severity of the ischemia, the size of the tissue that is ischemic, and the method of delivery. In one embodiment of the methods described herein, the therapeutically effective amount of cells is a safe and effective amount. In another specific embodiment, the amount of each cell type is at least 1×10⁴ cells. In another embodiment, the amount of each cell type that is administered to the subject is between about 10⁴ and about 5×10⁸ cells. The amount of cells administered to the subject will depend on the mode of administration and the site of administration. For example, a therapeutically effective cell dose via intracoronary injection (or intra-renal or intra-carotid or into coronary veins) may be lower than that for intra-femoral injection. When two types of cells are administered to the subject, such as when CD133+ cells and mesenchymal stem cells are administered, the ratio of the two cell types may be, for example, from about 20:1 to about 1:20, from about 10:1 to about 1:10, from about 5:1 to about 1:5, or from about 2:1 to about 1:2.

In one embodiment of the methods described herein wherein both a conditioned media composition and cell populations are administered to the subject, the conditioned media composition is administered to the subject in combination with the cells. In other embodiments, the composition may be administered to the subject before, concurrently, or after the administration of the cells.

In some embodiments of the methods described herein, at least one biological factor, such as a drug, polypeptide or nucleic acid, is further administered to the subject either as part of the conditioned media composition or separately. In one embodiment, the biological factor comprises a growth factor, a chemokine, a cytokine or an antibody. In one embodiment, the biological agent is selected from the group consisting of anti-rejection agents, analgesics, anti-oxidants, anti-apoptotic agents, erythropoietin, anti-inflammatory agents, anti-tumor necrosis factor α, anti-CD44, anti-CD3, anti-CD154, p38 kinase inhibitor, JAK-STAT inhibitors, anti-CD28, acetaminophen, cytostatic agents, Rapamycin, and anti-IL2 agents.

In preferred embodiments, the biological factor polypeptide promotes angiogenesis, vasculogenesis, or both. Exemplary angiogenic factors include, but are not limited to, basic fibroblast growth factor, acidic fibroblast growth factor, vascular endothelial growth factor, angiogenin, transforming growth factor α and β, tumor necrosis factor, angiopoietin, platelet-derived growth factor, placental growth factor, hepatocyte growth factor, and proliferin.

In another embodiment, the drug, agent or polypeptide is a thrombolytic agents, which include, but are not limited to, urokinase plasminogen activator, urokinase, streptokinase, inhibitors of α2-plasmin inhibitor, and inhibitors of plasminogen activator inhibitor-1, angiotensin converting enzyme (ACE) inhibitors, spironolactone, tissue plasminogen activator (tPA), an inhibitor of interleukin 1β converting enzyme, anti-thrombin III, and the like.

In another embodiment, the biological factor is a growth factor selected from the group consisting of erythropoietin, TGF-β1, TGF-β2, TGF-β3, BMP-2, BMP-4, BMP-6, BMP-12, BMP-13, fibroblast growth factor-1, fibroblast growth factor-2, platelet-derived growth factor-AA, platelet-derived growth factor-BB, platelet rich plasma, IGF-I, IGF-II, GDF-5, GDF-6, GDF-8, GDF-10, vascular endothelial cell-derived growth factor, pleiotrophin, endothelin, nicotinamide, glucagon like peptide-I, glucagon like peptide-II, Exendin-4, retinoic acid, parathyroid hormone, tenascin-C, tropoelastin, thrombin-derived peptides, laminin, biological peptides containing cell-binding domains and biological peptides containing heparin-binding domains. In one preferred embodiment, the biological factor is erythropoietin.

In another specific embodiment, the biological factor comprises a therapeutically effective amount of platelet microparticles. Platelet microparticles are described in U.S. Pat. No. 5,185,160 and in Nomura S, and Fukuhara S. Methods Mol Biol. 2004;272:269-77 and in Kim et al. Br J Haematol. February 2004;124(3):376-84. In one embodiment, the microparticles are autologous to the subject.

The therapeutically effective amount of the cell populations can be suspended in a pharmaceutically acceptable carrier or excipient. Such a carrier includes but is not limited to basal culture medium plus 1% serum albumin, saline, buffered saline, dextrose, water, and combinations thereof. The formulation should suit the mode of administration.

In a preferred embodiment, the composition of cells is formulated in accordance with routine procedures as a pharmaceutical composition adapted for intravenous administration to human beings. Typically, compositions for intravenous, intra-arterial, coronary vessel or intracardiac administration are solutions in sterile isotonic aqueous buffer. Where necessary, the composition may also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. When the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A variety of means for administering cells to subjects will, in view of this specification, be apparent to those of skill in the art. Such methods include injection of the cells into a target site in a subject. Cells may be inserted into a delivery device which facilitates introduction by injection or implantation into the subjects. Such delivery devices may include tubes, e.g., catheters, for injecting cells and fluids into the body of a recipient subject. In a preferred embodiment, the tubes additionally have a needle, e.g., a syringe, through which the cells of the invention can be introduced into the subject at a desired location. In a preferred embodiment, cells are formulated for administration into a blood vessel via a catheter (where the term “catheter” is intended to include any of the various tube-like systems for delivery of substances to a blood vessel). The cells may be prepared for delivery in a variety of different forms. For example, the cells may be suspended in a solution or gel. Cells may be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Modes of administration of the cells include but are not limited to systemic, intracardiac, intracoronary, intravenous or intra-arterial injection and injection directly into the tissue at the intended site of activity. The preparation can be administered by any convenient route, for example, by infusion or bolus injection and can be administered together with other biologically active agents. Administration may be systemic or more preferably at the intended site of activity. In cases when a subject suffers from global ischemia, a systemic administration such as intravenous administration, is preferred.

In one embodiment, more than one cell population is administered in conjunction with the conditioned media composition. In another embodiment the cell populations are administered before, at the same time, or after the administration of the composition. In some embodiments of the methods described herein, the cells which are to be administered to the subject are incubated in a buffer, such as a saline buffer. In another embodiment, the cells are incubated or propagated in the conditioned media itself or in components thereof prior to administration to the subject. In another embodiment, the buffer comprises human blood serum isolated from the subject. Human serum may be isolated using standard procedures. A solution comprising human blood serum may also be used to thaw a sample of cells that has been cryopreserved. In some embodiments, the solution comprising human serum contains between 1-20% human serum, or more preferably between 5-15%.

In embodiments of the methods described herein, administering the cells to the subject comprises an infusion of cells into the subject. The infusion may comprise a systemic infusion of cells into the subject, or it may comprise an infusion of cells in the proximity of the ischemic tissue, so as to facilitate the migration of cells to the ischemic tissue. The infusion may also be performed on the blood vessels that supply blood to the ischemic tissue, or to blood vessels which remove blood from the ischemic tissue. In specific embodiments of the methods described herein, the infusion of cells into the subject comprises an infusion into bone marrow, an intra-arterial infusion, an intramuscular infusion, an intracardiac infusion, and intracoronary infusion, an intravenous infusion or an intradermal infusion. In one embodiment of the methods described herein, the cells are administered to the subject by infusion into at least one coronary artery or coronary vein. In a specific embodiments of the methods described herein, the coronary artery is an epicardial vessel that provides collateral blood flow to the ischemic myocardium in the distribution of a chronic totally occluded vessel. In some embodiments of the methods described herein, administration of the cells to the subject is performed using an intra-arterial catheter, such as but not limited to a balloon catheter, or by using a stent. Any method currently available for delivering cells to a subject may be used.

In particular, the invention methods described herein for inducing neovascularization are useful for therapeutic vasculogenesis for the treatment of myocardial ischemia in humans. Administration of the compositions described hereon for the induction of neovascularization can be used as a sole treatment or as an adjunct to surgical and/or medical treatment modalities. For example, the methods described herein for treatment of myocardial ischemia can be used in conjunction with coronary artery bypass grafting or percutaneous coronary interventions. The methods described herein are particularly useful for subjects that have incomplete revascularization of the ischemic area after surgical treatments and, therefore, have areas of ischemic but viable myocardium. Subjects that can significantly benefit from the therapeutic vasculogenesis according to the methods of the invention are those who have large areas of viable myocardium jeopardized by the impaired perfusion supplied by vessels that are poor targets for revascularization techniques. Other subjects that can benefit from the therapeutic vasculogenesis methods are those having vessels of small caliber, severe diffuse atherosclerotic disease, and prior revascularization, in particular bypass grafting. Therefore, the therapeutic vasculogenesis according to the methods of the invention can particularly benefit subjects with chronic myocardial ischemia.

In one embodiment, the therapeutically effective amount of the cells that are optionally coadministered with the conditioned media compositions is a maximum number of cells that is safely received by the subject. Because the preferred injection route is intracoronary in the case of cardiac ischemia, and cells in culture may become larger than those originally isolated, the maximum dose should take into consideration the size of the vessels into which the cells are infused, so that the vessels do not become congested or plugged. The minimum number of cells necessary for induction of new blood vessel formation in the ischemic myocardium can be determined empirically, without undue experimentation, by dose escalation studies. For example, such a dose escalation could begin with approximately 10⁴/kg body weight of cells alone, or in combination with approximately 10⁴/kg of a second cell type. Effective amounts of cells sufficient to cause the desired neovascularization can be done based on animal data using routine computational methods. In one embodiment the effective amount is about 1.5×10⁵ cells per kg body mass to about 3×10⁵ per kg body mass. In another embodiment the effective amount is about 3×10⁵ per kg body mass to about 4.5×10⁵ cells per kg body mass. In another embodiment the effective amount is about 4.5×10⁵ per kg body mass to about 5.5×10⁵ cells per kg body mass. In another embodiment the effective amount is about 5.5×10⁵ per kg body mass to about 7×10⁵ cells per kg body mass. In another embodiment the effective amount is about 7×10⁵ per kg body mass to about 1×10⁶ cells per kg body mass. In another embodiment the effective amount is about 1×10⁶ per kg body mass to about 1.5×10⁶ cells per kg body mass. In one embodiment the effective amount of human cells is between about 1.5×10⁶ and 4.5×10⁶ cells per kg of the subject's body mass and In a preferred embodiment the effective amount is about 5×10⁵ cells per kg of the subject's body mass.

In some embodiments of the methods described herein, the composition comprising the cells is introduced into a vessel of the subject without substantially altering the arterial pressure. In other embodiments, the composition is introduced into a vessel by blocking arterial flow for an amount of time, such as from five seconds to two minutes, such that the injected cells can pool and adhere to the vessel. In one embodiment, a balloon catheter is used to allow pressure driven administration.

V. Methods of Providing Compositions and Products

One aspect of the invention provides methods of providing therapeutic products derived from umbilical cord blood for use by health care professionals for the treatment of a disorder in a subject. In a preferred embodiment, the therapeutic products do not comprise cells, whereas in another embodiment they do not comprise live cells. In one preferred embodiment, the therapeutic products comprise conditioned media compositions or related devices as described in the preceding sections.

One aspect of the invention provides a composition for use by health care professionals for the treatment of a disorder in a subject, the method comprising: (a) providing a sample of umbilical cord blood; (b) culturing at least one cell-type from the umbilical cord blood in a cell culture medium to generate conditioned media; (c) concentrating or isolating at least one component of the conditioned media and formulating a pharmaceutical composition which comprises said component; (d) packaging the composition under sterile conditions; and (e) distributing the package for use by health care professionals for treating the disorder in the subject. In one embodiment, the cell-type that is cultured is selected from the group consisting of an AC133+ cell, a CD34+ cell, a mesenchymal stem cell, a MAPC and an EPCs. In some embodiments, the pharmaceutical composition is incorporated into a device, such as a sustained delivery device (e.g. a stent), prior to packaging. In one embodiment, the method further comprises billing the subject or the subject's insurance carrier for the composition.

In one embodiment, the sample of umbilical cord blood is autologous to the subject. In another embodiment, the umbilical cord blood is allogenic to the subject. In another embodiment, the umbilical cord blood is not HLA-matched with the subject. In one embodiment, the umbilical cord blood sample is from a single umbilical cord. In another embodiment, the umbilical cord blood is from a plurality of umbilical cords. In a related embodiment, the pharmaceutical product is generated by culturing at least one cell type from a plurality of umbilical cord blood samples.

In one embodiment the methods provided herein for providing compositions to health care professional, the disorder is ischemia. In a specific embodiment, the ischemia is selected from the group consisting of limb ischemia, ischemic cardiomyopathy, myocardial ischemia, cerebrovascular ischemia, renal ischemia, pulmonary ischemia and intestinal ischemia. In a preferred embodiment, the ischemia myocardial ischemia.

In one embodiment, the disorder is not cancer. In a related embodiment, the subject is not afflicted with cancer, while in another embodiment the subject has never been diagnosed with cancer. In one embodiment, the disorder is not a hematopoietic disorder. In a related embodiment, the subject is not afflicted with a hematopoietic disorder, while in another embodiment the subject has never been diagnosed with a hematopoietic disorder. In another embodiment, the subject is not in need of hematopoietic reconstitution. In another embodiment, the subject is not in a chemotherapy patient.

In one embodiment, the cell-type that is cultured is genetically modified to express a transgene, such as a cytokine, chemokine, growth factor, antibody, adhesion factor, extracellular matrix protein or an integrin. Preferred transgenes include PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof. In one embodiment, the transgene is VEGF or TGFβ1 or both.

In some embodiments, step (a) comprises (i) providing a sample of umbilical cord blood; and (ii) isolating a cell type from sample of umbilical cord blood. In a specific embodiment, isolating the cell population comprises (i) contacting the sample of umbilical cord blood with an affinity agent. In one embodiment, the affinity agent is an antibody, a fragment thereof, a polypeptide immobilized on a substrate such as a bead or plastic surface, or the like. In a preferred embodiment, the affinity agent comprises and antibody or fragment thereof which binds to the cell type, whereas in another embodiment the antibody or fragment thereof does not bind to the cell type. Exemplary antibodies are those which bind to CD34, CD133, CD45, CD117, CD105, CXCR1-4, FGFR1, FGFR2, VEGFR1, VEGFR2, SH2, SH3 or SH4. Affinity-based techniques for the isolation of a cell type from a population of cells are well-known in the art. Other methods of isolation specific cell types include culturing the cell populations on substrates to which cells preferentially adhere.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

The contents of any patents, patent applications, patent publications, or scientific articles referenced anywhere in this application are herein incorporated in their entirety.

Exemplification

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other stem cell sources and selection methods, other culture media and culture methods, other dosage and treatment schedules, and other animals and/or humans, all without limitation, can be employed, without departing from the scope of the invention as claimed.

### EXAMPLE 1

### Isolation and Characterization of Endothelial Precursor Cells from Umbilical Cord Blood and Adult Bone Marrow

Mononuclear cells were isolated from umbilical cord blood (UCB) or adult bone marrow (BM) and placed in short-term culture under conditions supportive of the development of endothelial precursor cells (EPC). Adherent cells recovered from the cultures were found to exhibit EPC characteristics, as analyzed using multiple in vitro assays, including cytochemistry, flow cytometry, microscopic morphology and immunostaining.

1) Isolation of Cells

Mononuclear cells (MNC) from fresh UCB or BM were isolated using density gradient centrifugation. EPC cells were isolated expanded in cell culture according to the method of Kalka et al. (2000) PNAS 97: 3422-3427. Briefly, the MNC were plated on human fibronectin coated tissue culture flasks at a density of 4-6×10⁶ cells/ml (UCB MNC) or 1-2×10⁶ cells/ml (BM MNC) in EC basal medium-2 (EBM-2) (Clonetics, San Diego) with 5% fetal bovine serum (FBS) and standard SingleQuo™ additives that included human VEGF-1, human fibroblast growth factor-2 (FGF), insulin-like growth factor-1 (IGF-1), hydrocortisone, ascorbic acid and heparin. Non-adherent cells were removed by washing with phosphate-buffered saline (PBS) after 4 days of culture and the medium was changed every fourth day thereafter. During the second week of culture, the adherent cells adopted the spindle-like morphology characteristic of EPCs.

At day 6-7, cells were trypsinized and counted. The yield of adherent cells from UCB cultures was, on average, 2.5%±0.4% of the initial MNC input, compared to a yield of 21.5%±3.7% obtained from BM MNC.

2) Cellular Staining of Adherent Cells for EPC Characteristics

a) Two principal cytochemical staining features of mature endothelial cells are the adherence of specific lectin proteins, such as Ulex europaeus agglutinin (UEA)-1, and the uptake of acetylated low-density lipoprotein (acLDL). Fluorescent microscopy of adherent cells was performed to detect dual binding of FITC-labeled UEA-1 (Sigma) and 1,1′-dioctadecyl-3,3,3′,3′-tetramethylindocarbocyanine (DiI)-labeled acLDL (Biomedical Technologies, Stoughton, Mass.).

Adherent cells were first incubated with acLDL at 37° C. and fixed with 1% paraformaldehyde for 10 min. After washes, the cells were reacted with UEA-1 (10 μg/ml) for one hour. After staining, samples were viewed at 40× with a confocal microscope set to record total cell fluorescence.

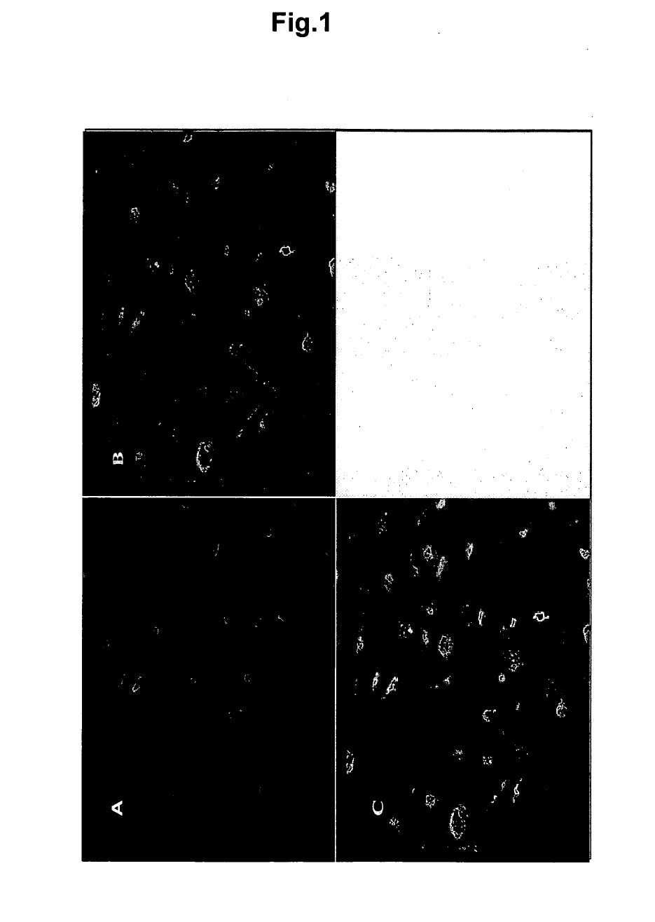

FIG. 1 illustrates fluorescent microscopy images showing cytochemical staining of UCB-derived EPC. It was found that the majority of the cells exhibited uptake of acLDL (A). A smaller proportion exhibited positive staining for UEA-1 lectin (B). Composite dual staining results for both cytochemical stains simultaneously are displayed in C. Cells demonstrating double-positive fluorescence were identified as differentiating EPCs.

A comparison of the uptake of acLDL and morphology of EPC cells derived from both BM and UCB was determined. During the second week of culture, cells derived from both sources displayed uptake of acLDL and exhibited similar morphologic features (data not shown).

b) von Willebrand factor (vWF) is a well-characterized multimeric glycoprotein synthesized by vascular endothelial cells and megakaryocytes. Adherent cells cultured from UCB were stained for vWF. Slides with surface adherent cells were fixed in room temperature acetone for 10 min. and air dried. The cells were then reacted with a polyclonal rabbit anti-human Factor VII related antigen commercially available from Dako (Carpinteria, Calif.). Detection of cells binding the antibody was achieved using routine horse-radish peroxidase labeled streptavidin-biotin technology (LSAB2, Dako) and 3,3-diaminobenzidine as the chromogen. Staining was viewed by phase contrast microscopy using a magnification of 40×.

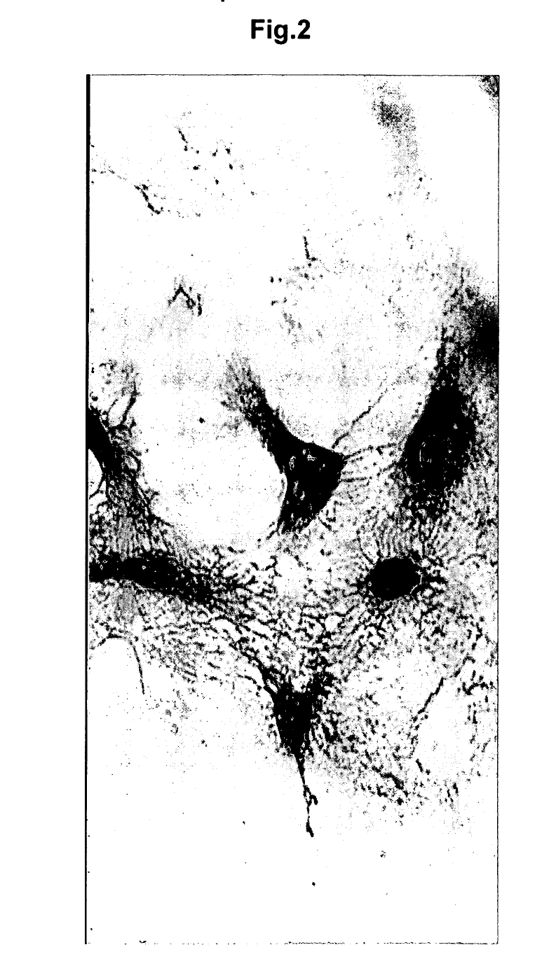

As illustrated in FIG. 2 the non-selected adherent cells cultured from UCB exhibited a distinct endothelial staining pattern. The brown perinuclear stain is due to immunoperoxidase conjugated to secondary antibodies that are reacting with perinuclear vWF particles. Human umbilical vein endothelial cells (HUVECS) were stained as positive controls, and fibroblasts as negative controls (data not shown).

3) Flow Cytometry Analysis of EPC Cells Derived from UCB

a) Selection and Phenotyping of CD133⁺ Cells:

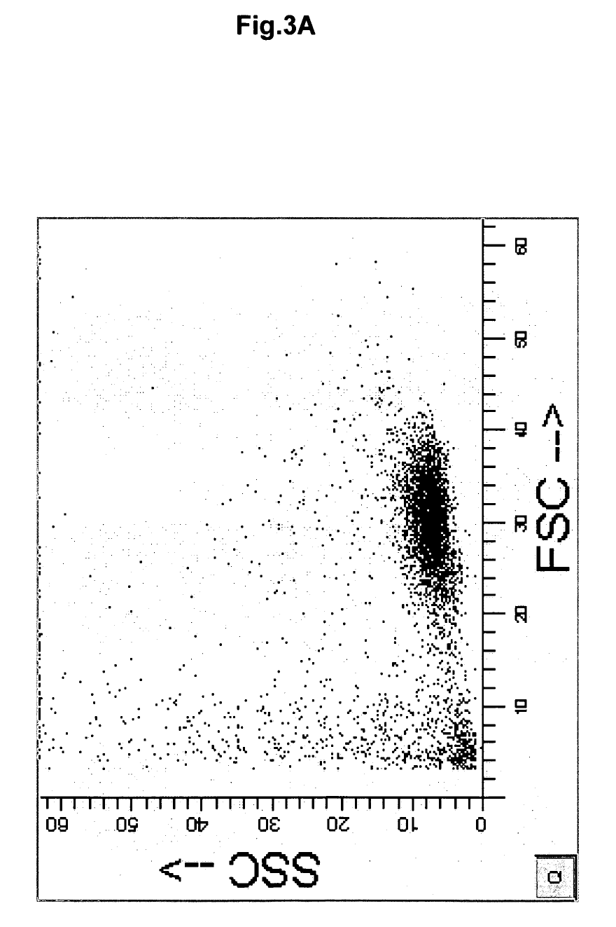

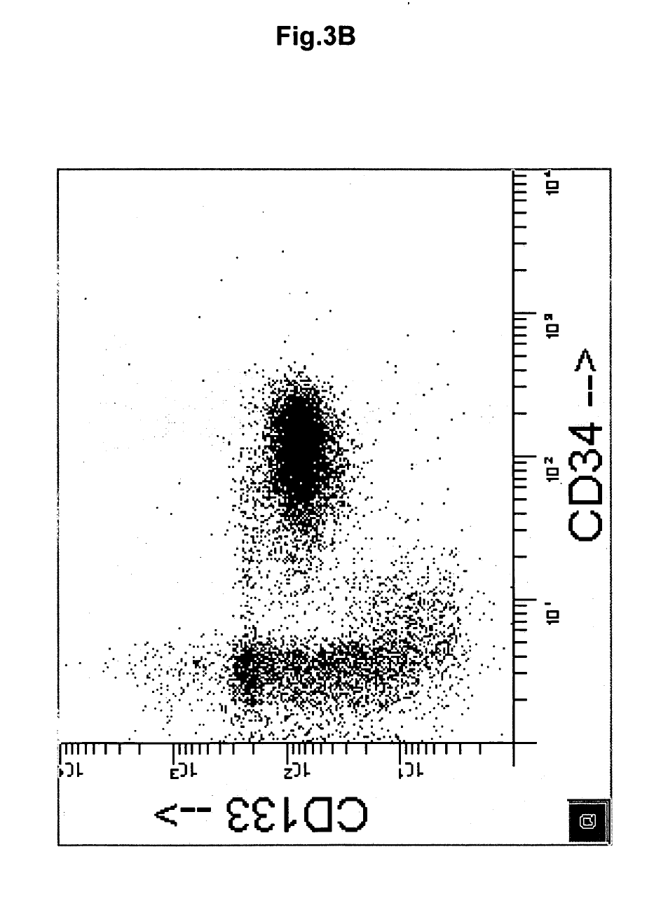

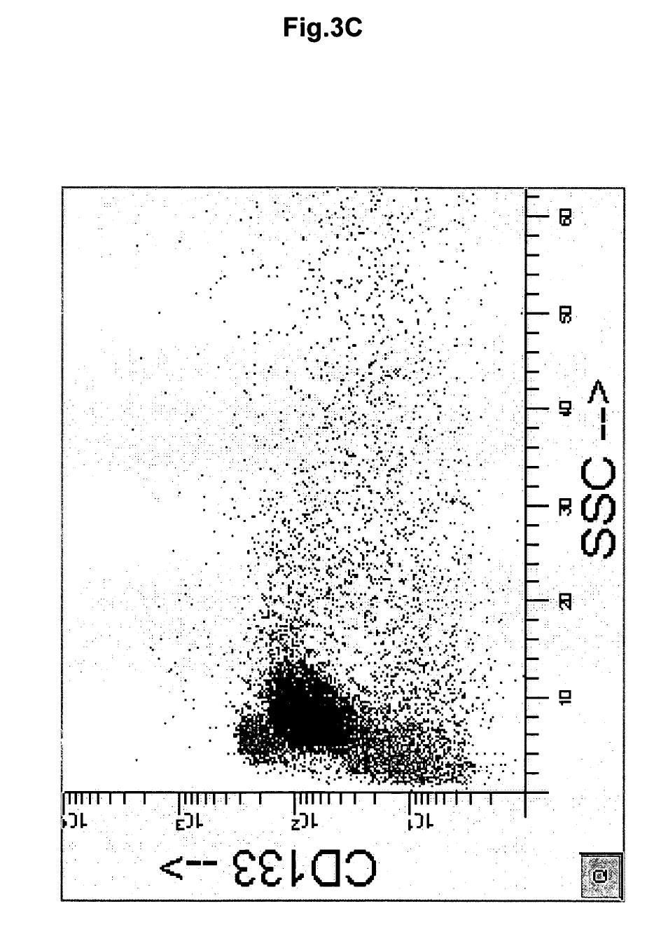

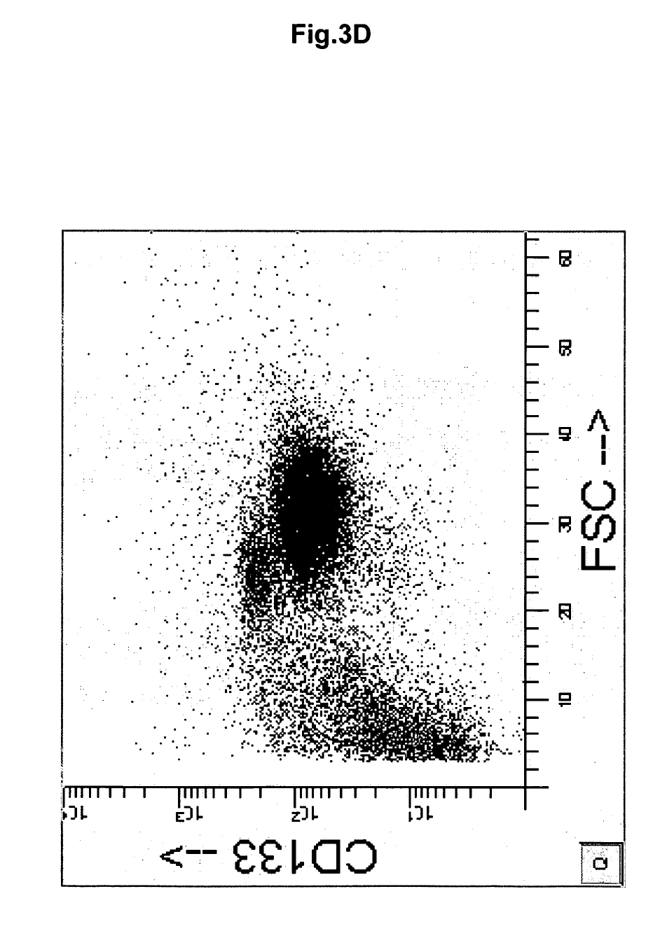

50×10⁶ MNC from UCB were labeled with magnetic bead-conjugated anti-CD133 antibody (Miltenyl) and passed through two consecutive magnetic columns to yield 0.1×10⁶ of positively selected CD133⁺ cells. The selected CD133⁺ cells were characterized by flow cytometry and staining for CD34 and CD133. FIG. 3 illustrates distinctly identified populations of CD133⁺/CD34⁻ cells (100) and CD133⁺/CD34⁺ cells (200), as displayed versus size (Forward Scatter, FSC) and granularity (refractivity Side Scatter, SSC). FSC gain was increased for better resolution of very small cells. No gating was applied.

b) Phenotyping of Unselected EPC Cells Derived from UCB and BM:

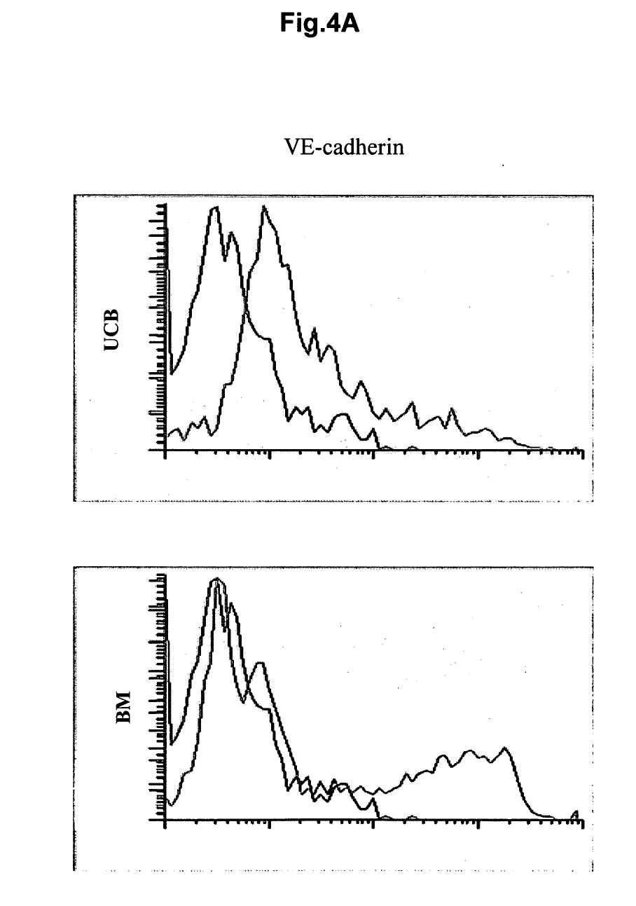

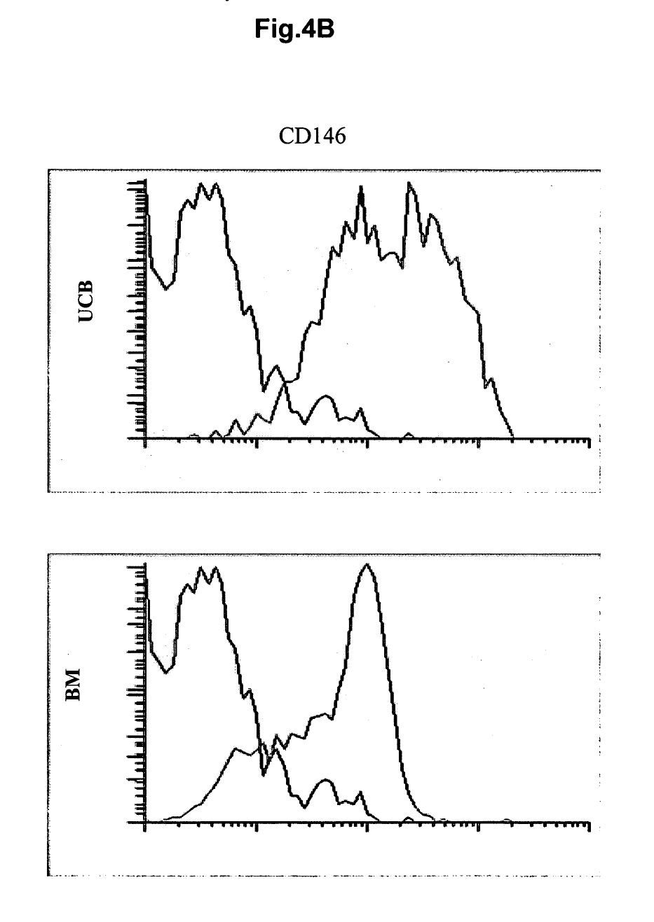

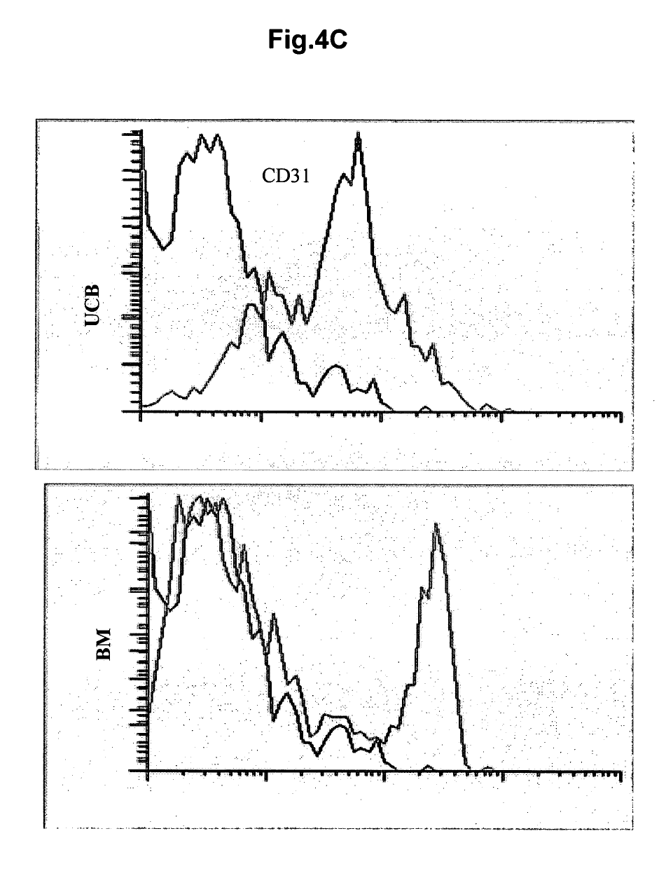

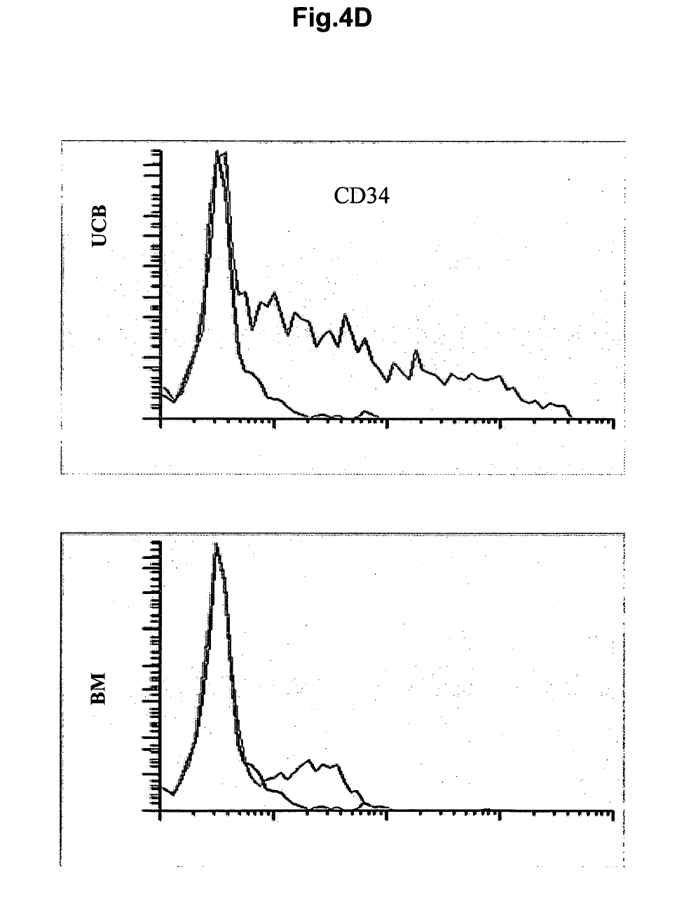

UCB cells were cultured for 19 days and BM cells were cultured for 12 days in EMB-2 media. Adherent cells were trypsinized and stained for CD34 and mature endothelial-specific markers CD146 (P1H12, MUC18 or MCAM), CD31 and human vascular endothelium (VE)-cadherin. As illustrated in FIG. 4, over 60% of the cultured adherent cells were positive for CD146. Expression of CD31 was 25% in BM derived EPC, compared to 50% in UCB derived cells. However, CD31 staining was brighter in BM. VE-cadherin was expressed in 10% of cells from BM compared to 24% in the cells from UCB. EPC derived from UCB showed expression of CD34 in 25% of cells, compared to 10% of the BM derived EPC.

In summary, the foregoing studies demonstrated that non-selected UCB and BM cells rapidly proliferate and expand under endothelial cell culture conditions. These UCB and BM derived EPC exhibit multiple endothelial characteristics.

### EXAMPLE 2

### Transplantation of UCB and BM-Derived EPC in an in Vivo Model

In vivo studies of neovascularization in a murine hind limb ischemia model, in NOD/SCID mice, were performed. The results illustrate that UCB is an optimal source of EPC. Although UCB lacks stromal elements present in BM, EPC from UCB demonstrated an equivalent biological effect in the in vivo model to that exerted by EPC derived from BM sources.

1) Treatment Groups. All procedures were performed in accordance with Case Western Reserve University's Institutional Animal Care and Use Committee. NOD/SCID mice, age 10-15 weeks and weighing 20-25 grams were used. Prior to surgery, the mice were irradiated with 2.5 Gy from a Cesium-137 source to further reduce rejection of injected human cells. The mice were fasted over night but allowed free access to water. They were then anesthetized with intraperitoneal injection of a combination of ketamine and pentobarbital. Under sterile conditions, a small skin incision was made in right groin area. The right femoral artery was exposed, ligated along with adjacent branches (with #000 silk) and transected. Special care was given not to ligate the femoral vein and femoral nerve. The skin incision was then closed with continuous suture fashion (#000 silk). After femoral artery ligation, the mice were divided into four groups. Group 1 animals received an intracardiac injection of 1×10⁶ (in 0.02 ml of media) adherent (EPC) UCB cells harvested at day 7 of culture. Group 2 animals received intracardiac injection of 1×10⁶ of adherent (EPC) BM cells harvested at day 7 of culture. Group 3 and Group 4 animals similarly received 0.02 ml. of complete EBM-2 medium or saline alone, respectively. Immediately after surgery and injection of cells, baseline blood blow of both the ischemic right leg and the non-operated left leg was measured using a laser Doppler flowmeter (Laser flowmeter ALF21D, Advance Company LTD, Tokyo, Japan). Laser Doppler measurements were repeated at 7 days, 14 days and 28 days after the surgery. A ratio of perfusion in the ischemic/healthy limb was used to compare neovascularization in the three study groups.

2) Comparison of Perfusion Ratios in Animals Treated with EPC from UCB or BM

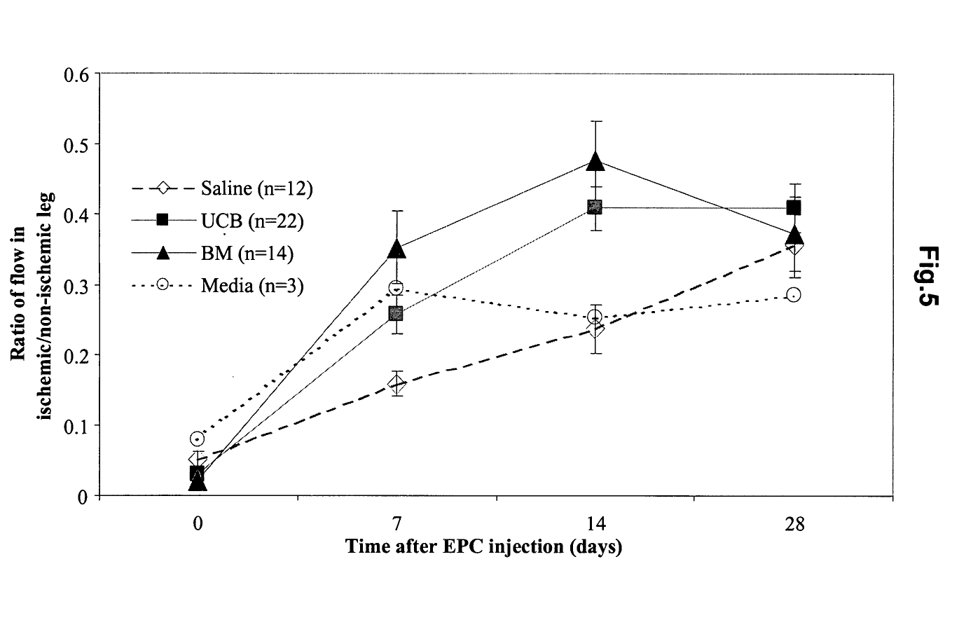

FIG. 5 illustrates a comparison of the perfusion ratio between the ischemic and non-ischemic leg. Immediately following femoral ligation the perfusion ratios were 0.057±0.011 (control group injected with EBM-2 medium only), 0.029±0.007 (UCB-derived EPC) and 0.020±0.004 (BM-derived EPC) showing reduced perfusion in all groups. After 14 days, there was a statistically significant higher blood flow in the injured leg in study groups receiving UCB-derived EPC compared to the control group and between the BM-derived EPC group and the control group (p<0.001). Perfusion ratios in the control group remained low, with a ratio of 0.24±0.032 (n=14), compared to a ratio of 0.41±0.031 (n=22) in the group receiving UCB-derived EPC (p=0.0008) and a ratio of 0.48±0.039 (n=14) in the group receiving BM-derived EPC. At day 14 there was no significant difference in the ratios between the two sources of EPCs (p=0.18). Subsequent measurements at time point 28 days were notable for improvement in Doppler blood flow in control animals rendering perfusion ratios equalized when comparing the control group and mice receiving cell infusions.

3) Histological Assessment of Ischemic Hind-Limb in Treatment Groups

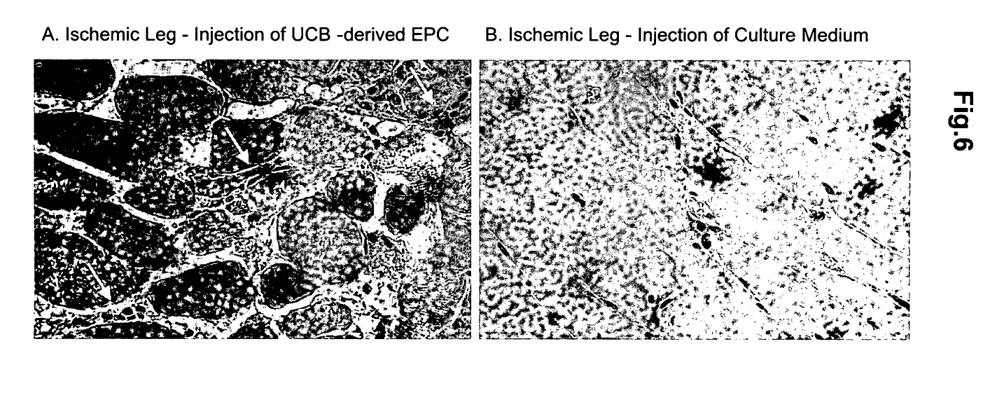

Tissue from the lower calf muscle of both hind limbs was harvested at day 28 for histological evaluation. The samples were fresh frozen in liquid nitrogen and fixed in formalin. Frozen sections of 6 μm thickness were mounted on saline-coated glass slides and stained using immunohistochemistry techniques to identify incorporation of EPCs derived from human cells by staining with anti-human CD31 antibody. As illustrated in FIG. 6, specimens from mice that were injected with UCB EPCs showed positive staining for CD31, where the control mice injected with complete EMB2 medium did not. Healthy limbs of all groups did not show positive CD31 staining (data not shown). The specimens from the BM EPC-injected mice showed similar results (data not shown).

### EXAMPLE 3

### Selection and Purification of CD133⁺ Cells from UCB

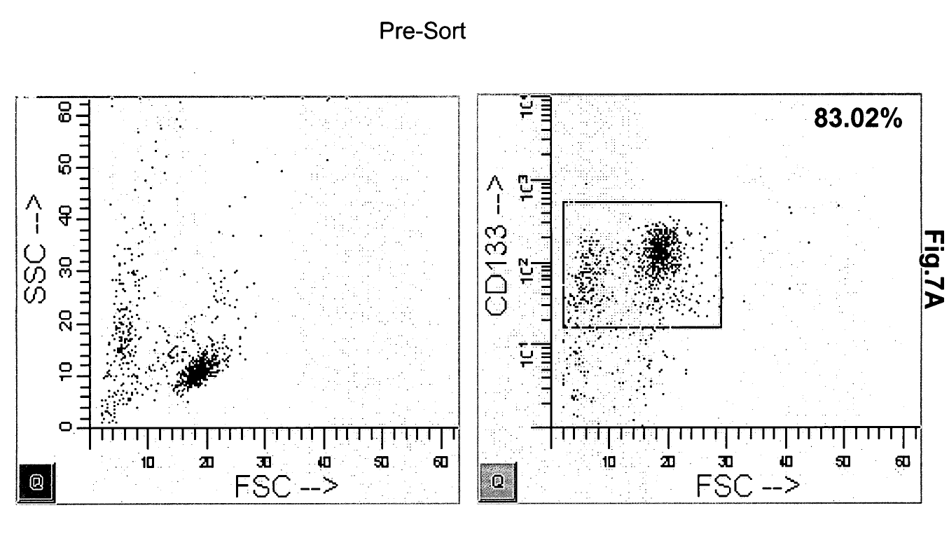

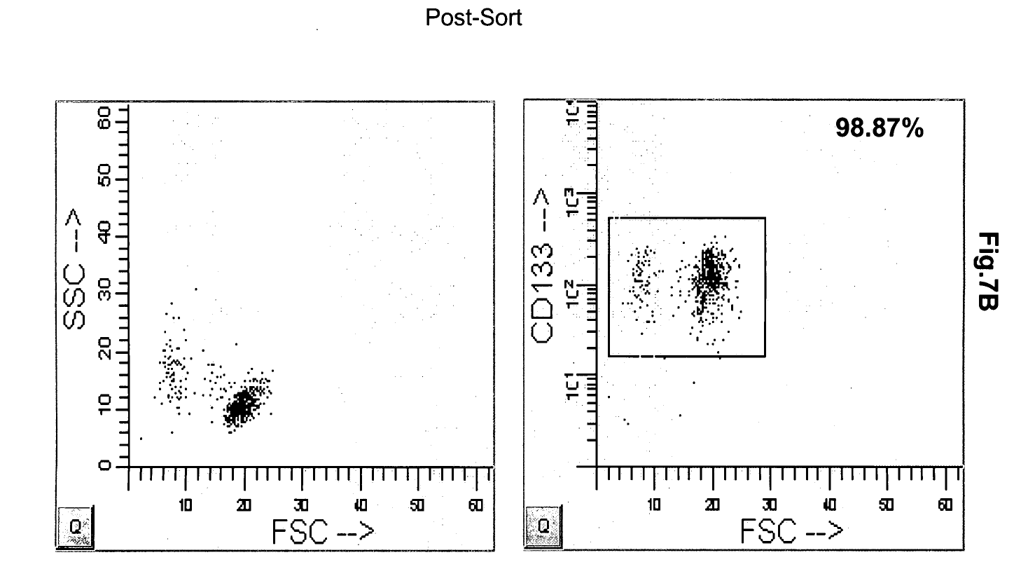

For isolation and purification of CD133⁺ cells, mononuclear cells were isolated from UCB as described above and were labeled with CD133⁺ conjugated magnetic beads, followed by automated sorting through magnetic columns (Automacs, Miltenyi). By passaging the labeled cells through a single column, the routine yield was 0.4% of the original MNC, with a purity of CD133⁺ cells ranging between 75% and 85%. By passage of the MNC through two consecutive magnetic columns, the purity could be raised to 91.2% CD133⁺ cells, but the yields dropped to 0.2%. Further purification attempts were made by fluorescence-activated cell sorting (FACS). CD133⁺ cells were isolated by passage through one magnetic column, stained with CD133-phycoerythrin (PE)-conjugated antibody and further purified by FACS. As illustrated in FIG. 7, the resulting purity after passage through one magnetic column was 83.02% CD133⁺ cells. After FACS, the purity was increased to 98.87%, with a final yield of 0.1% of the initial MNC input.

### EXAMPLE 4

### Culture-Expansion and Characterization of Purified CD133⁺ Cells

1) Flow Cytometry Analysis of Surface Markers of CD133⁺ Cells in Endothelial Cell-Driving Cytokines or Hematopoietic Cell-Driving Cytokines

Purified CD133⁺ cells isolated according to Example 3 were cultured either in hematopoiesis-driving cytokines or in cytokines that have been reported to generate endothelial cells from CD133⁺ cells. (Gehling, U. M. et al. Blood 95(10): 3106-3112.) Briefly, for hematopoiesis-driving conditions, the CD133⁺ cells were plated on a 96-well plate at a concentration of 0.2×10⁶ cells/well/condition and incubated for 24 hours in either medium alone (Iscove's Modified Dulbecco's Medium, IMDM) with 2% FBS, or in hematopoietic culture medium (IMDM), 30% FBS, 50 ng/ml of stem cell factor (SCF), 20 ng/ml of human granulocyte-macrophage colony stimulating factor (GM-CSF), granulocyte colony stimulating factor (G-CSF), interleukin-3 (IL-3), IL-6, and 3 U/ml of erythropoietin). For endothelial cell-driving conditions, 0.2×10⁶ CD133⁺ cells were similarly plated and incubated in endothelial culture medium (IMDM, 10% FBS, 10% horse serum 1 mM hydrocortisone, 100 ng/ml of stem cell growth factor (SCGF), and 50 ng/ml of VEGF). After 24 hours of incubation, the cells were analyzed by flow cytometry for the hematopoietic surface markers CD34 and CD45, as well as for expression of BCL-2 and p21, which are cell cycle and apoptosis-regulating proteins, respectively, shown to play a role in regulation of the fate of HSC. For example, p21^{clp1/waf1} is an inhibitor of cyclin-dependent kinases and mediates cell cycle arrest in G1. It has been shown that in p21^{clp1/waf1} deficient mice there is increased proliferation of HSC under normal homeostatic conditions and exhaustion of the stem cell pool, suggesting that p21^{clp1/waf1} may be a molecular switch governing the entry of HSC into the cell cycle. Over expression of the anti-apoptotic protein BCL-2 in the hematopoietic compartment of transgenic mice has been shown to improve numbers of HSC as well as in vitro plating capacity, and maintained HSC in a more quiescent cell cycle status.

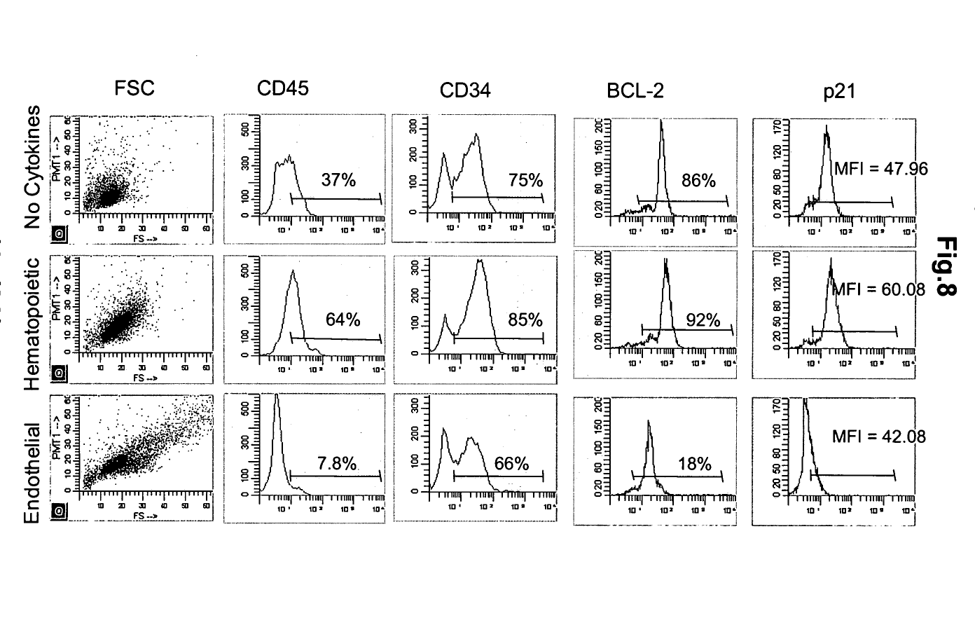

The results of flow cytometry, illustrated in FIG. 8, show the intensity of expression in the total cells expressed as mean fluorescence intensity (MFI) or percentage of total cells analyzed. CD45 and CD34 expression were strongly increased after 24 hours of culture in hematopoiesis-lineage specific cytokines. CD45 expression was lost in endothelial cytokines, suggesting that the cells have already started differentiation away from the hematopoietic lineage. Expression of both p21 and BCL-2 proteins was increased in hematopoietic cytokine conditions. However, expression of both proteins decreased significantly in endothelial cytokine conditions, again suggesting that the two cell populations have already started differential gene expression programs.

2) Cell Cycle Analysis in Freshly Isolated or 24 Hour Cultured CD133⁺ Cells from UCB.

Cell cycle stages were analyzed in CD133⁺ cells freshly isolated as in Example 3, as well as in CD133⁺ cells after 24 hours of culture in medium alone, or under hematopoietic- or endothelial-driving conditions, as described above, or under hematopoietic conditions for 72 hours. Cells were fixed, permeabilized, and DNA stained with Hoechst under standard conditions, and analyzed for cell cycle stages.

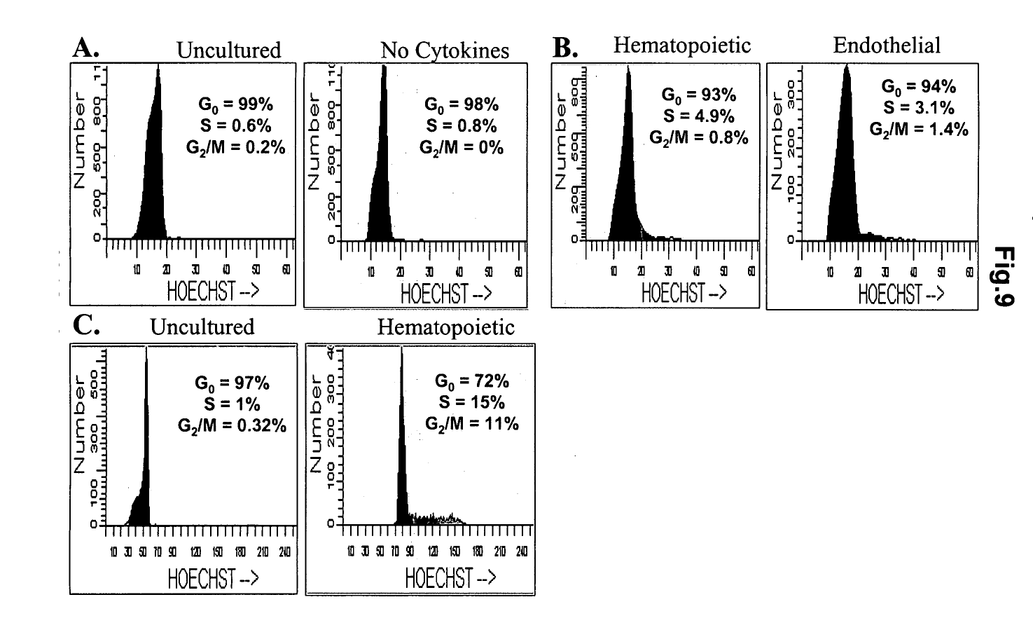

The results are illustrated in FIG. 9. The analysis of cell cycle stages of freshly isolated CD133⁺ cells (A) showed that 99% of the cells were resting in G₀ phase. After 24 hours of culture in cytokines (B), no significant cell division was found in hematopoietic or endothelial conditions, with the majority of the cells (93%-94%) still in G₀ phase at that time. After 72 hours in hematopoietic conditions, however, 15% of the cells were in S-phase and 11% of the cells were in G₂/M-phase. This data shows that differential protein expression, discussed above, after only 24 hours of incubation in specific cytokines, was progressing along differential gene expression programs, although very little cell division had taken place at that time. Therefore, with no cellular division having occurred at 24 hours, cells cultured in hematopoietic or endothelial conditions are still, in effect, the same cells as originally plated.

### EXAMPLE 5

### Neovascularization in the Mouse Hind-Limb Injury Model by EPC Derived from Purified UCB CD133⁺ Cells

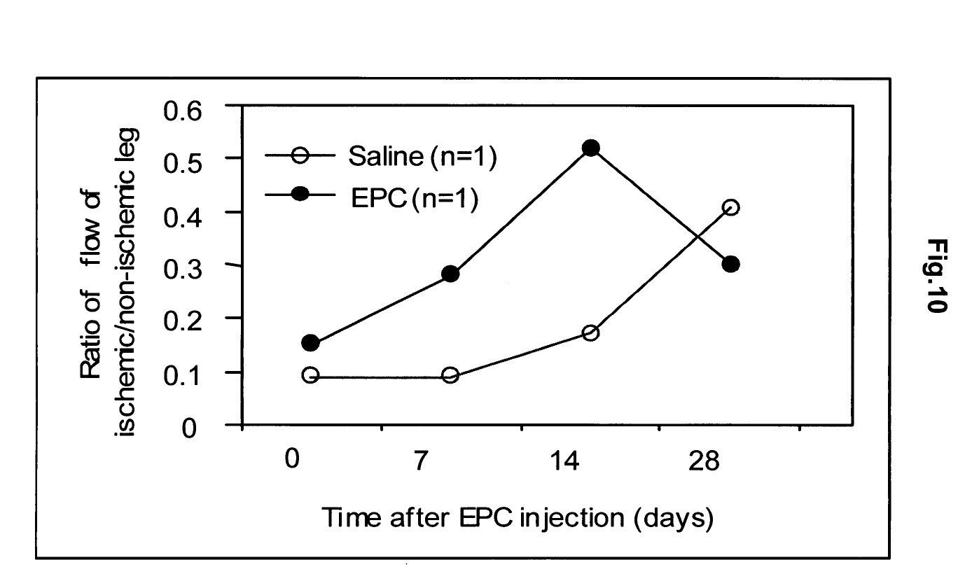

CD133⁺ cells were selected as described in Example 3. After selection, the cells were seeded at 50,000-70,000 cells/well in 96-well plates under the same endothelial-driving culture conditions as described in Example 4. After 7 days of culture, cells were injected intracardially into mice that had undergone hind-limb femoral artery ligation by the method described in Example 2. Cell yields ranged from 58-130% of plated CD133⁺ cells, or 0.26% of the initial number of MNC. Blood flow was measured by laser Doppler flowmeter over time, and the results illustrated in FIG. 10 are expressed as the ratio between the blood flow in the injured and the uninjured leg over time. The results show increased blood flow in the mouse receiving CD133⁺ cells 14 days after surgery, when compared to the saline control injected on the same day. Analyses at a later time point (day 28) were notable for a significant improvement in the Doppler flow measurements in control mice injected with saline alone.

### EXAMPLE 6

### Human Mesenchymal Stem Cells and Human Umbilical Vein Endothelial Cells Reciprocally Induce Mitotic Expansion

Early angiogenic interactions between cells that are not in physical contact are mediated by soluble factors. Human mesenchymal stem cells secrete factors to support developmental processes such as osteogenesis, hematopoiesis and osteoclastogenesis. Many of the cytokines that modulate these processes also affect endothelial cell growth. The following examples illustrate that hMSCs secrete proteins that stimulate growth of mature endothelial cells. The examples also illustrate that soluble factors derived from mature endothelial cells stimulate the growth of hMSCs.

1) Human Bone Marrow-Derived Mesenchymal Stem Cells (hMSC): Isolation and Culture-Expansion

Bone marrow was aspirated from the iliac crests of six human donors. Human mesenchymal stem cells were purified and cultured by a modification of previous reported methods (Haynesworth, S E et al. 1992. Bone 13, 81-88). Briefly, bone marrow aspirates were transferred from 20 ml. syringes into 50 ml conical tubes containing 25 ml of growth medium. Growth medium consisted of Dulbecco's Modified Eagles' Medium supplemented to 10% (v/v) with fetal bovine serum (FBS, GIBCO, Gaithersburg, Md.) from screened and selected lots. The tubes were spun in a Beckman table-top centrifuge at 1,200 rpm in a GS-6 swinging bucket rotor for 5 minutes to pellet the cells. The fat layer and supernatant were aspirated with a serological pipette and discarded. Cell pellets were resuspended to a volume of 5 ml with growth medium and then transferred to the top of preformed 35 ml gradients of 70% Percoll. The samples were loaded into a Sorvall SS-34 fixed angle rotor and centrifuged in a Sorvall High Speed Centrifuge at 460 g for 15 minutes. The low density fraction of approximately 12 ml (pooled density=1.03 g/ml) was collected from each gradient and transferred to 50 ml conical tubes to each of which was added 30 ml of growth medium. The tubes were centrifuged at 1,200 rpm to pellet the cells. The supernatants were discarded and the cells were resuspended in 20 ml of growth medium and counted with a hemocytometer after lysing red blood cells with 4% acetic acid. Cells were adjusted to a concentration of 5×10⁷ cells per 7 ml and seeded onto 100 mm culture plates at 7 ml per plate.

The cells were cultured in growth medium at 37° C. in a humidified atmosphere containing 95% air and 5% CO₂, with medium changes every 34 days. When primary culture dishes became nearly confluent at 10-14 days, the cells were detached with 0.25% (w/v) trypsin containing 1 mM EDTA for 5 min at 37° C. The enzymatic activity of tyypsin was stopped by adding ½ volume of calf serum. The cells were counted and resuspended in growth medium. Cell yield was about 0.26% of the initial number of MNC.

2) Conditioned Medium Growth Assays

Human mesenchymal stem cells, obtained as in Example 6 Part I, or human umbilical vein endothelial cells (HUVECs) were plated in 35 mm dishes and allowed to attach in growth medium. Following attachment, the cells were washed and then incubated for 12 hours in serum-free (hMSC) or low serum (HUVEC) medium to reduce residual serum proteins that might remain in the cytoplasm of the cells and synchronize growth phase of these cells. The cells were washed again before they were incubated for 72 hours (hMSCs) or 48 hours (HUVECs) in various concentrations of conditioned medium. Cells were quantified by hemocytometer.

To generate hMSC conditioned medium, hMSC at 75% confluence in 100 mm plates were washed and incubated in serum-free Dulbecco's Modified Eagles' Medium with low glucose (DMEM-LG) for 24 hours. The hMSCs were washed with Tyrode's balanced salt solution and then incubated to condition a serum-free defined medium (80% Iscove's, 12% DMEM-LG, and 8% chick fibroblast basal medium MCDB 201) for 72 hours. After the conditioning period, the medium was removed and centrifuged to remove cellular debris. The cells that conditioned the medium were quantified and conditioned medium was normalized to the cell number by dilution with serum-free defined medium to 10,000 cells/ml.

Conditioned medium was concentrated to 20× using Centricon 3 KDa molecular weight (MW) cut-off centrifugal devices in a Sorvall centrifuge at 4° C. Concentrated conditioned medium and filtrate (flow-through from concentration units containing no protein over 3 KDa MW) were either used immediately or stored at −20° C. The filtrate was centrifuged to remove cellular debris and then used to dilute the 20× conditioned medium to 2× (twice the final concentration). To produce 1× conditioned medium, fresh serum-free medium was added at a 1:1 ratio to provide essential nutrients.

HUVEC-conditioned medium was prepared as described above for hMSC conditioned medium, except that the HUVECs were grown in Medium 199 with 1% FBS for 48 hours. After concentration, the HUVEC-conditioned medium was diluted to 2× with flow through filtrate, as described above. The conditioned medium was then diluted to 1× with fresh Medium 199 with 1% FBS.

3) Effect of Conditioned Medium on Mitotic Expansion of hMSCs or HUVECs

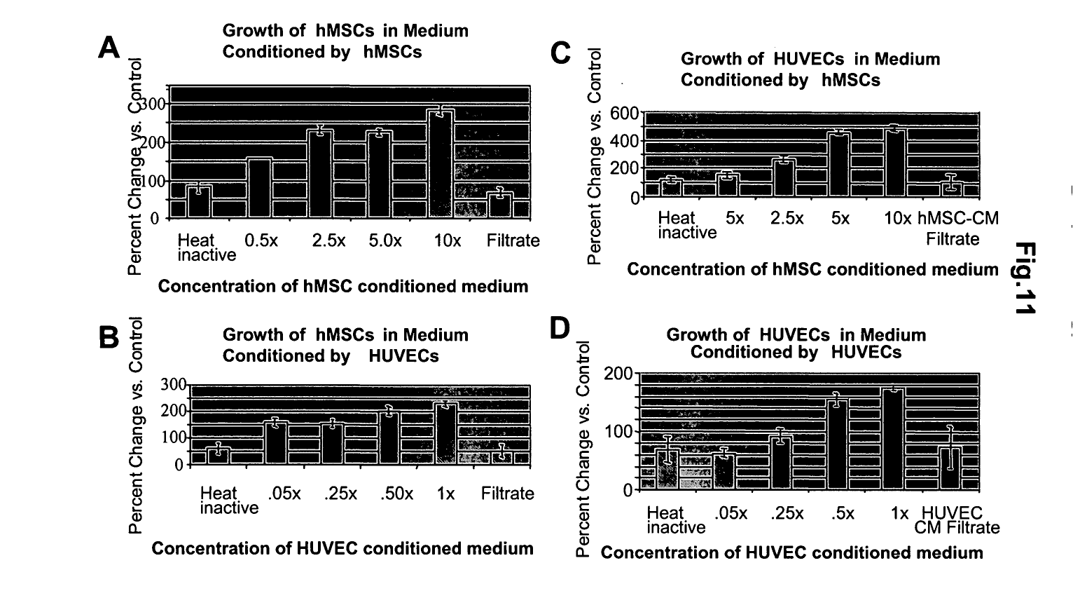

FIG. 11 illustrates the dose response mitotic expansion of hMSC cell number following incubation in medium conditioned by HUVECs (B), and the dose response mitotic expansion of HUVEC cell number following incubation in medium conditioned by hMSCs (C), respectively. The growth stimulatory effect by the conditioned medium (CM) was not evident with conditioned medium that had been heat inactivated by boiling. Filtrates (flow through from concentration units with a 3 KDa MW cut-off) did not have a stimulatory effect for either cell type.

Control medium in all figures was combined unconditioned medium at a 1:1 ratio with fresh minimal medium best suited for the cell type. HUVEC 1× control medium contains 1% FBS. Dilutions of HUVEC control medium contain proportionately less FBS but do not vary by more than 1% FBS. FIG. 11(A) and 11(D) are control growth cultures.

### EXAMPLE 7

### Chemotactic Migration of hMSCs and HUVECs Toward Secreted Factors in Conditioned Medium

Tissues acquire new vasculature, in part, through the release of factors that induce the chemotactic migration of endothelial cells from existing blood vessels into the tissue. Likewise, newly formed vasculature matures and stabilizes, in part, as a result of their interaction with mesenchymal pericytes that migrate to the site of the new vessel in response to chemotactic factors released by the endothelial cells. The following example illustrates that hMSCs can stimulate endothelial cell migration, serve as pericyte precursors, and respond to chemotactic factors released by endothelial cells. Boyden chambers were used to measure the migration of hMSCs and HUVECs in response to chemotactic factors secreted into the conditioned medium of the other.

1) Chemotactic Migration Toward Conditioned Medium in Boyden Chambers

Lower wells of Neuroprobe 48-well Boyden chambers were loaded with varying concentrations either the hMSC- or HUVEC conditioned medium described in Example 6. A 1% gelatin coated polycarbonate membrane with 5 μm pores was placed on top of the lower wells and the chamber was assembled. hMSCs or HUVECs were pelleted and washed thoroughly before they were suspended in either serum-free (for dose response assays) or varying concentrations of conditioned medium (checkerboard assays). hMSC or HUVEC cell suspensions were loaded in the upper wells. The chambers were incubated at 37° C. for 5 hours to permit migration of cells from the upper well, through the membrane, into conditioned medium in the lower wells. Following the 5 hour incubation, the chambers were disassembled and the membrane was removed. Cells were scraped from the upper surface of the membrane leaving only cells that migrated through the membrane pores. The migratory cells were then fixed in formaldehyde, stained with crystal violet, and mounted on slides. Slides were scanned for dose response and quantified by direct cell count using an Olympus 480E microscope. A row of three dots on the filter represents migration of cells in three wells of a given condition.

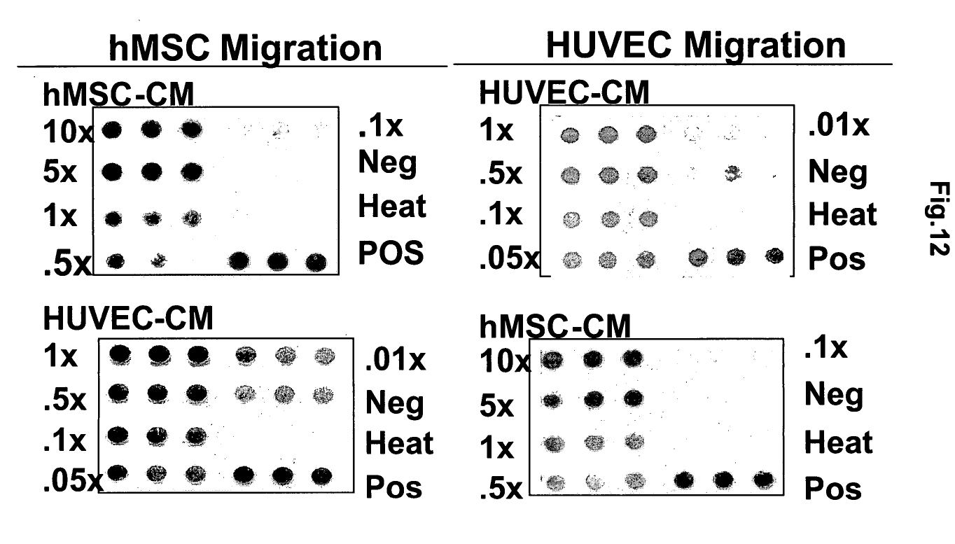

FIG. 12 illustrates migration of hMSCs (top) and HUVECs (bottom) toward hMSC-conditioned medium (left panel), and migration of HUVECs (top) and hMSCs (bottom) toward HUVEC-conditioned medium (right panel). For both cell types, the greatest migration is observed in the three spots on the upper left hand corner of the membrane that correspond with the highest concentration (10×) of hMSC- or HUVEC-conditioned medium, respectively. The intensity of the spots (that directly corresponds to the number of cells attached to the membrane) decreases as the concentration of conditioned medium decreases, thus demonstrating a dose dependent migration of both hMSCs and HUVECs toward HUVEC- or hMSC-conditioned medium, respectively. Heat denatured conditioned medium showed migration patterns similar to the negative control. 10% FBS was used as a positive control.

### EXAMPLE 8

### Human Mesenchymal Stem Cells Express Vascular Endothelial Growth Factor (VEGF) Genes and VEGF Receptor Genes

VEGFs have been described as endothelial cell-specific ligands with receptors found exclusively on endothelial cells. However, recent reports demonstrate expression of VEGF receptors on non-endothelial cells including human bone marrow stromal cells. The following two examples demonstrate that hMSCs also express VEGF growth factors and receptors.

1) RT-PCR Analysis of the Expression of VEGF Family of Growth Factors mRNA by hMSC.

RT-PCR was used to show messenger RNA expression of VEGF family growth factor genes. Qiagen kits were used to generate total RNA from pelleted hMSCs. A cDNA synthesis kit (Amersham) generated cDNA from total RNA. cDNA was combined with specific primers for VEGF family genes (VEGF-A, -B, -C, -D, and PIGF) and added to RT-PCR Ready-To-Go beads for amplification in a Robocycler 480 PCR machine. All reactions employed the same 35 cycle amplification program with optimal annealing temperatures set for the specific primer.

2) Visualization of VEGF PCR Products

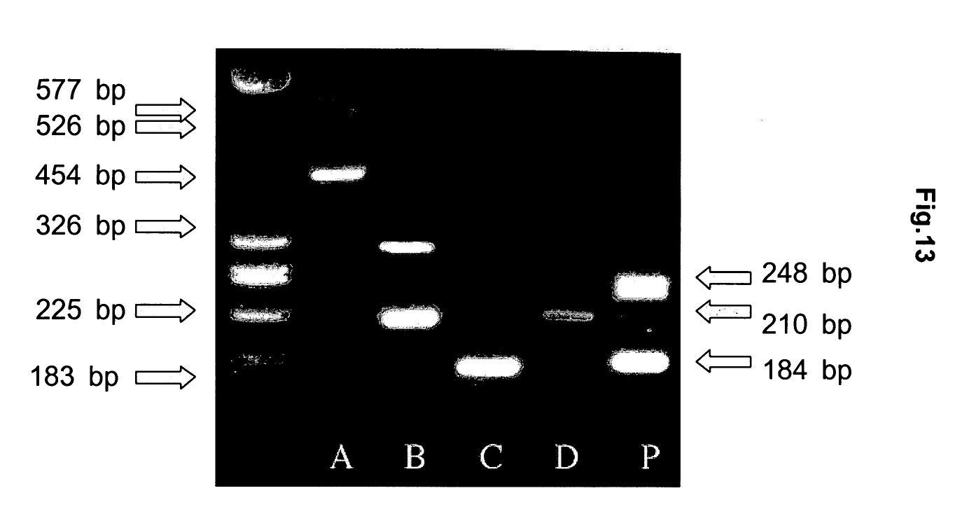

Varying amounts of PCR product were run on a 2% agarose gel and visualized using ethidium bromide staining. FIG. 13 illustrates the sizes of the isolated PCR products, as follows: VEGF-A at 577 bp, 526 bp, and 454 bp; VEGF-B at 326 bp and 225 bp; VEGF-C at 183 bp; VEGF-D at 225 bp; and PIGF at 248 bp and 184 bp.

3) RT-PCR Analysis of VEGF Receptor Expression by hMSC

RT-PCR analysis was performed as described in Example 9 using specific primers for VEGF receptors 1, 2 and 3, as well as Neuropilin-1 and Neuropilin-2.

4) Visualization of VEGF PCR Receptor Products

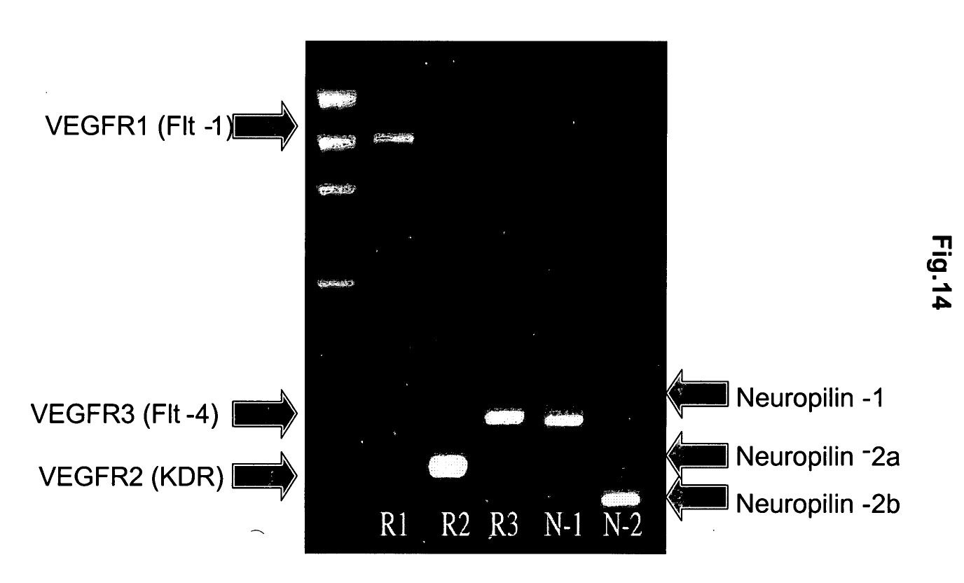

The visualization was carried out as described above. FIG. 14 illustrates high molecular weight DNA markers, VEGFR1 (1,098 bp), VEGFR2 (326 bp), VEGFR3 (380 bp); Neuropilin-1 (375 bp) and Neuropilin-2 (304 bp and 289 bp).

### EXAMPLE 9

### Direct Cell Contact Between Pericyte Precursors and Endothelial Cells Leads to Interactions that Activate TGF-β1, which Ends the Angiogenic Growth Phase and Induces Vascular Differentiation of Each Cell Type

TGF-β1 is secreted in a latent form by most cells in culture. The physiological relevance of TGF-β1 is the regulation of its activation. There are no reports in the literature of production of active TGF-β1 in non-transformed cells in monoculture. However, co-cultures of endothelial cells with a multipotent murine fibroblast (10T1/2 cells), pericytes, or smooth muscle cells in co-culture with endothelial cells, have been shown to activate latent TGF-β1 through a mechanism involving proteolytic cleavage of a latency peptide by plasmin. This example illustrates that hMSCs interact with endothelial cells through direct cell contact and activate the key anti-angiogenic factor, TGF-β1. ELISA analysis was employed to detect active TGF-β1 protein in conditioned medium from hMSC and HUVEC monocultures or co-cultures, prepared as described in Example 6, above.

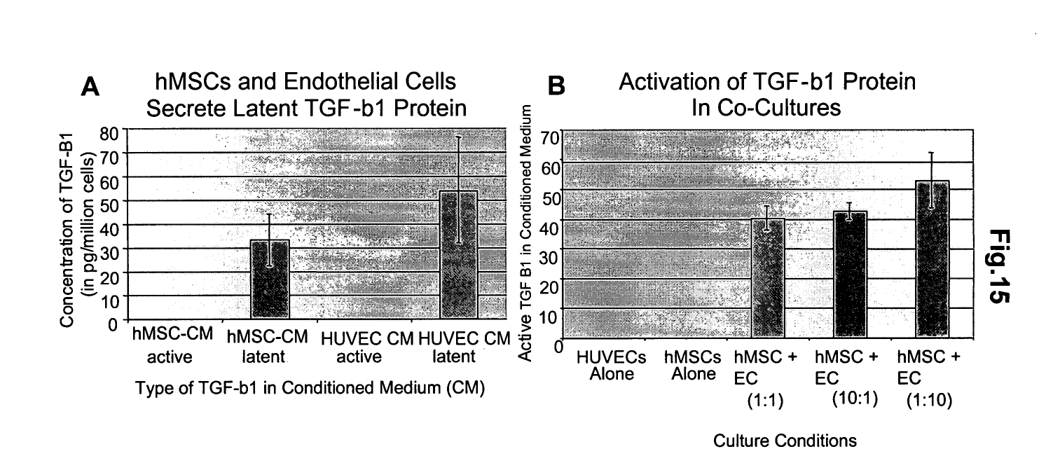

FIG. 15(A) demonstrates secretion of latent TGF-β1 by hMSCs and endothelial cells in monoculture. As expected, no active TGF-β1 was measurable in conditioned medium from hMSC or HUVECs in monoculture. FIG. 15(B) demonstrates that active TGF-β1 was not produced in monocultures of hMSCs or HUVECs but was measured in co-cultures of the same cells.

### EXAMPLE 10

### hMSCs Selectively Migrate to Endothelial Tube-Like Structures

Evidence suggests that endothelial cell tubes recruit surrounding mesenchymal cells to migrate towards and co-localize with newly forming vessels to stabilize them. Endothelial cell tubes in 3-dimensional type I collagen gels are an in vitro correlate of newly formed vessels. The data presented in the examples above demonstrate that hMSCs and HUVECs interact through secreted proteins that induce chemotactic migration. Further, the data demonstrate that hMSCs interact with HUVECs in co-culture and modulate signaling to activate TGF-β1, an anti-angiogenic factor that has been shown to end the angiogenic growth phase and induce terminal differentiation of certain fibroblasts and endothelial cells.

This example demonstrates that hMSCs can be induced to migrate to endothelial cell tube-like structures, co-localize, and differentiate into pericytes.

1) Preparation of Tube-Like Structures and Visualization of hMSC Migration

Briefly, DiI stained hMSCs were added to Vitrogen (type I collagen) 3D gel cultures of endothelial cell tube-like structures to investigate co-localization. DiI is a vital dye. To establish cultures of HUVEC tube-like structures, HUVECs were plated at 300,000 cells/ml onto 1% gelatin coated 35 mm plates. Following attachment, endothelial growth medium was removed and cells were washed thoroughly with Tyrode's solution. A solution of Vitrogen gel at a 1:1 ratio with DMEM-LG with 10% FBS was added to the endothelial cells. Following solidification of the Vitrogen mixture, an additional 1 ml of endothelial growth medium was added and cultures were incubated overnight to permit tube-like structure formation.

To stain hMSCs with DiI, hMSCs were plated at 50,000 cells/ml in 35 mm plates. hMSCs were incubated overnight in DMEM-LG with 10% FBS to permit attachment. Cultures were then washed with Tyrode's solution and incubated for 6 hours in DMEM-LG with 10% FBS combined with 1 μg/ml DiI. Following the incubation, hMSCs were washed thoroughly and then trypsinized to remove cells from the plate. The hMSCs were pelleted by centrifugation and then resuspended at 30,000 cells/ml in DMEM-LG with 2% FBS.

One ml of hMSC suspension was added to the upper surface of HUVEC tube-like structures in gel culture. Co-localization required migration of hMSCs through the 3D gel to tube-like structures located near the bottom surfaces. Cultures were monitored and photographed.

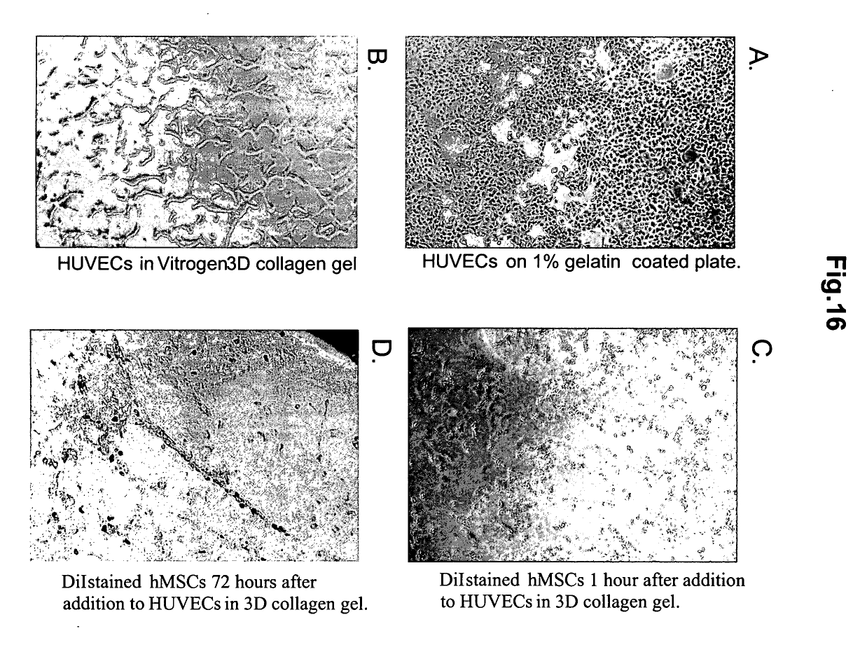

The results are illustrated in FIG. 16. In panel A, HUVECs are shown in a typical 2-dimensional culture. Panel B shows the tube-like structures that formed 12 hours after Vitrogen 3D collagen gel was added to the cells in panel A. An extensive network plexus of endothelial tubes is visible. Panel C illustrates the DiI stained hMSCs randomly distributed across the surface of the 3D collagen gel. Panel D shows the same culture 24 hours after addition of the hMSCs to the HUVECS in the 3D collagen gel. The hMSCs migrated through the gel and selectively co-localized with the endothelial cell tubes. Results were reproducible using multiple hMSC and HUVEC donors in the same experimental conditions.

### EXAMPLE 11

### Release of Angiogenic Factors by UCB CD133+ Cells

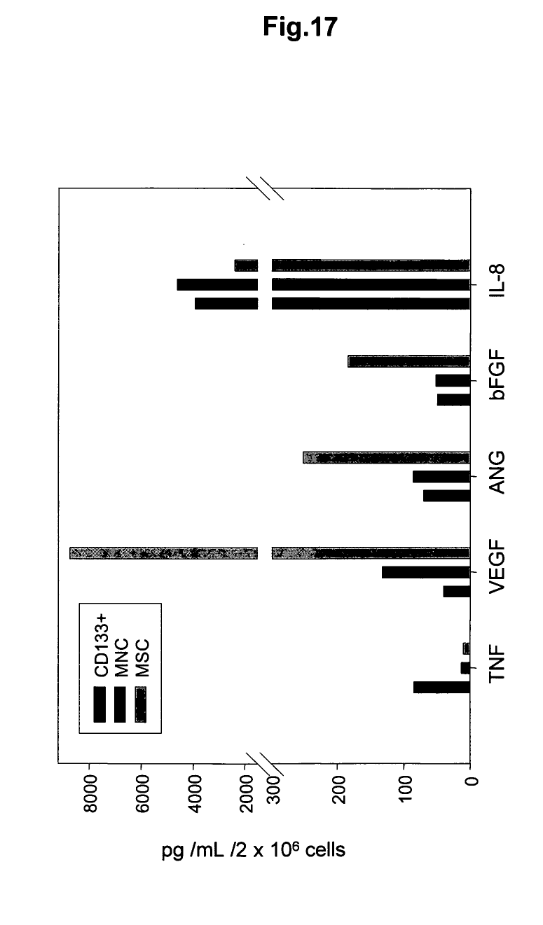

Applicants measured production of angiogenic proteins by MNC and CD133+ cell from UCB as well as huMSC generated from adult BM. Supernatant was collected from UCB MNC and CD133+ in addition to huMSC after 24 h in culture at cell concentration 2×106 cell/mL. Supernatants were analyzed for angiogenic factors including IL-8, basic fibroblast growth factor (bFGF), angiogenin (Ang), vascular endothelial growth factor (VEGF) and tumor necrosis factor (TNF). These factors were measured by angiogenesis cytometric bead array kit (BD Biosciences; San Diego, Calif.). To ensure all samples fell within detection limits, standard curves for non-diluted and 1:5 dilution samples were run for each sample. Samples were analyzed on an LSR (BD Biosciences) and evaluated using BD CBA Software v. 1.1 (BD Biosciences). Results shown in FIG. 17 demonstrate elevated levels of VEGF, Angiogenin and bFGF produced by huMSC compared to both CD133+ and MNC from UCB. Production of IL-8 was elevated in all three of the cell types analyzed.

### EXAMPLE 12

### Co-Localization of CD133 EPC to HUVEC-Derived Capillary-Like Networks on Matrigel

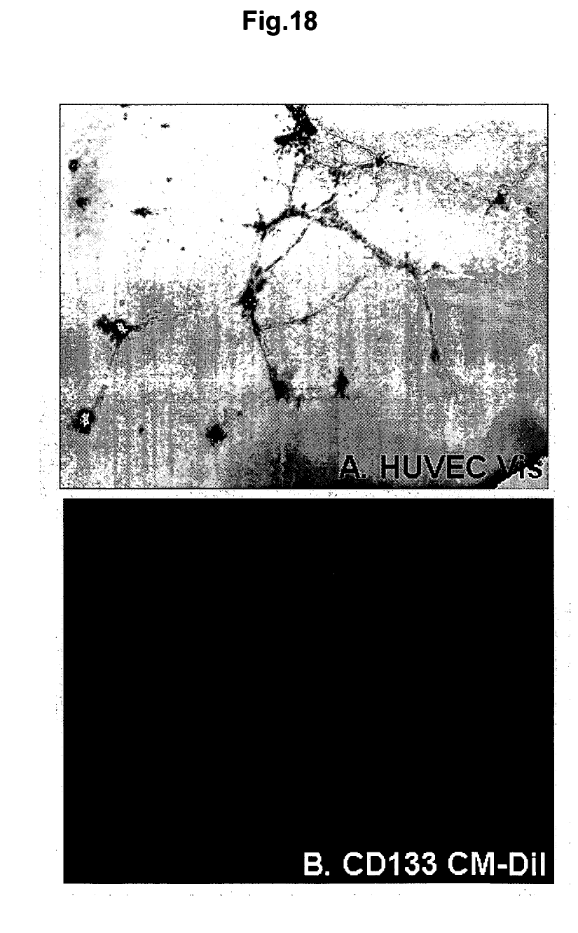

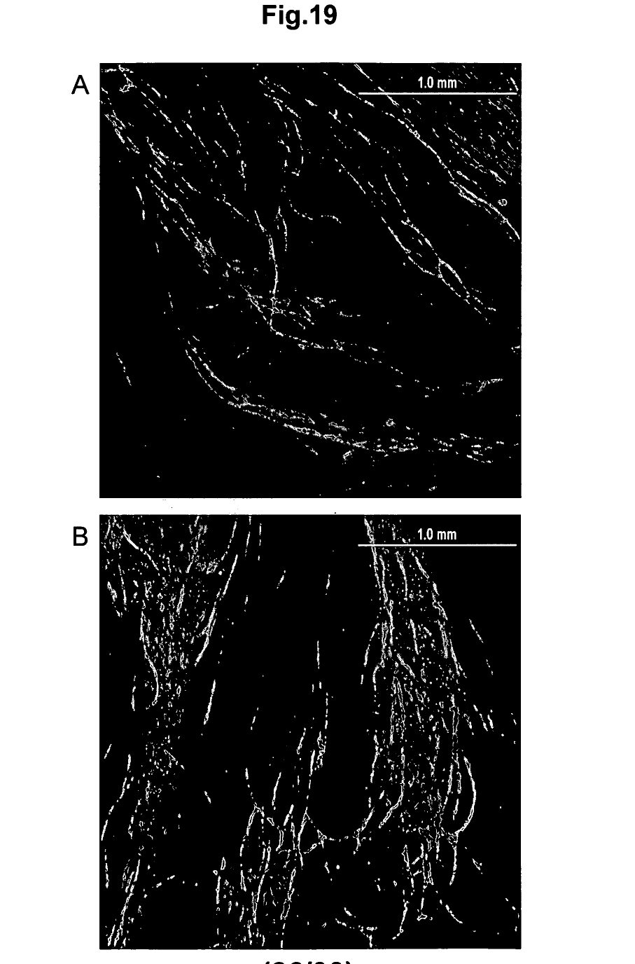

In tissue culture wells containing a thick preparation of Matrigel, an equivalent number of primary HUVEC (passage 1) and CM-DiI-labeled CD133+ cells were mixed. After 24 hours of culture at 37° C., digital camera images were acquired under both visible (A) and fluorescent lighting conditions (FIG. 18). CD133+ EPC were observed to localize with the capillary-like tubule structures at both branching points and along the tubules extensions.

### EXAMPLE 13

### Organotypic Culture Systems to Study HUVEC-Mesenchymal Stromal Cell Interactions

Our preliminary data demonstrated that bone marrow-derived mesenchymal stromal cells (huMSC) interact with HUVEC through secreted proteins that induce chemotactic migration (see FIG. 8 above). We have also demonstrated that huMSC interact with HUVEC in co-culture and modulate signaling to activate TGF-β1 (see FIG. 10). Further, CD133⁺ EPC associate with HUVECs on Matrigel (FIG. 12). We have utilized an organotypic assay for measuring the development of capillary-like tubules from committed human umbilical cord vein endothelial cells (HUVEC) in order to determine whether cellular interactions between CD133⁺ EPC and huMSC serve to augment capillary-like tubules from committed human umbilical cord vein endothelial cells (HUVEC). This organotypic assay utilizes a monolayer of adherent huMSC as a supportive matrix and as a cellular layer continuously producing cytokines and growth factors. Selected CD133⁺ EPC were co-cultured with equivalent numbers of HUVEC on a confluent layer of huMSC. After 14 days culture, capillary-like tubule structures were labeled with an antibody directed against an endothelial antigen CD31. In the presence of CD133⁺ cells, the area, perimeter and size of developed tubules was increased by 15.2-fold, 3.4-fold, and 3.2-fold, respectively demonstrating that the additional of CD133⁺ EPC enhances capillary-like tubule formation and the stimulates the proliferation of HUVEC.

### EXAMPLE 14

### Human MSCs Support Human Hematopoiesis in NOD.SCID Mice

To determine whether human MSCs support engraftment and survival of human hematopoietic stem cells in vivo, we infused 2, 4 or 8×106 human umbilical cord blood mononuclear cells with or without 1×106 unrelated human huMSC into sub-lethally (250 cGy) irradiated NOD.SCID mice. Red cell depleted, human umbilical cord blood (UCB) cells were obtained from the hematopoietic Stem Cell Facility. In mice receiving UCB cells alone, high levels of human engraftment was seen with 8×106 UCB cell dose (4-90%), whereas no engraftment was seen after infusion of 2×106 UCB cell dose. Only 2 out of 10 mice had detectable (>0.2%) but low levels (<1%) of human engraftment after infuision of 4×106 UCB cells. In contrast 8 out of 10 mice had human engraftment after co-infusion of 4×106 UCB cells plus 1×106 unrelated human huMSC (p=0.02 by Fisher Exact Test). None of the 4 mice had human engraftment after co-infusion of 4×106 UCB cells plus 1×106 immortalized mouse mesenchymal cells (BMC-9 line). Furthermore, the level of human engraftment was also significantly higher in mice co-infused with human huMSC (2.5±2% vs. 0.2±0.2%, two-tailed p=0.005, unpaired t test with Welch correction) compared to UCB alone.

### EXAMPLE 15

### HuMSC Suppression of Allogeneic Proliferation in Mixed Lymphocyte Reaction (MLR)

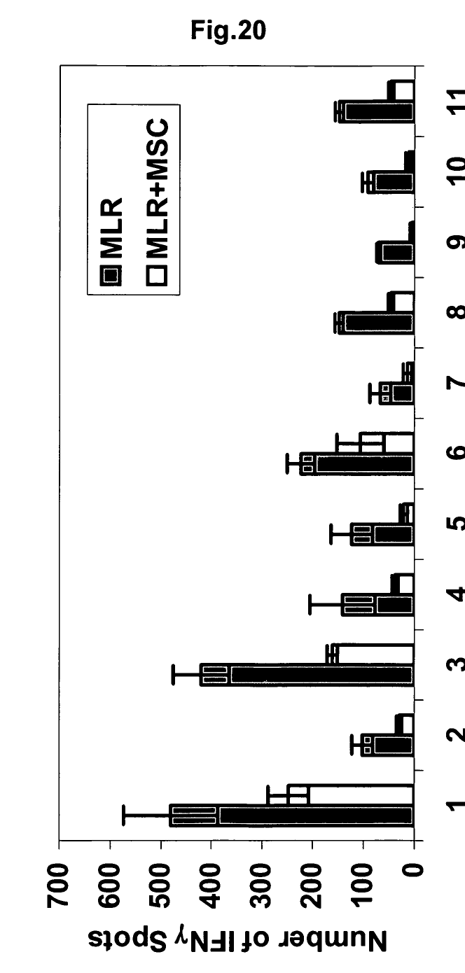

Applicants have conducted in vitro studies to determine normal immune cell (mononuclear cells, MNC) proliferative responses to human mesenchymal stem cells (huMSC). As outlined below in FIGS. 20 to 23, we have observed that huMSC inhibit activation of normal lymphocytes by soluble factors found in culture-conditioned medium. Importantly, we found that huMSC must be activated by CD14⁺ monocytes to exert these immunosuppressive effects. This reaction between huMSC and CD14⁺ monocytes appears to be mediated by IL-1β. Activated huMSC secrete TGFβ1 that is partially responsible for inhibition of normal allogeneic T-lymphocytes. Although huMSC stimulate IL-10 production in mixed lymphocyte reaction (MLR), IL-10 does not appear to contribute to huMSC-mediated T-lymphocyte inhibition. We observed expected up-regulation of T-lymphocyte activation antigens CD25, CD38, and CD69 after PHA stimulation. This up-regulation was significantly inhibited both in CD3⁺CD4⁺ and CD3⁺CD8⁺ lymphocytes when PHA stimulation was conducted in the presence of an adherent layer of huMSC obtained from an unrelated allogeneic donor.

We measured activation and IFN-γ secretion of human blood lymphocytes by allogeneic human blood MNCs (mixed lymphocyte reaction, MLR) using human-Interferon-γ Elispot assay and found consistent inhibition of this reaction by addition of huMSC derived from donors not related to either MLR donors (3^{rd} party). We evaluated the variability of this inhibition using huMSC prepared from 11 different donors and 11 independent MLRs (FIG. 20). A large variation was seen in the number of IFNγ spots formed per 300,000 blood MNC tested in each experiment (183±140 spots, range 67-480) due to the expected variation in the numbers of allo-reactive cells present in each specimen. Addition of huMSC resulted in a consistent inhibition of the MLR in every experiment. Despite a large variation in the number of spots observed in each MLR, the percent inhibition by huMSC was consistent between experiments (71±14%, range 48-92%). A direct relationship was seen between the number of alloreactive lymphocytes at baseline and the percent inhibition by huMSC. To determine the specificity of huMSC-mediated suppression of T-cell activation, rat MSCs, human dermal fibroblasts and murine NIH3T3 cells were tested in the same assay system. While unrelated human huMSC and xenogeneic rat huMSC did not elicit human T-cell activation, unrelated human dermal fibroblasts and murine NIH-3T3 cell elicited T-cell activation. Furthermore human dermal fibroblasts failed to suppress the mixed lymphocyte reaction. These results suggest that human huMSC exert an immunosuppressive effect on normal allogeneic lymphocytes.

### EXAMPLE 16

### T-Cell Inhibitory Function of huMSC Require an Activation Step by Blood MNC

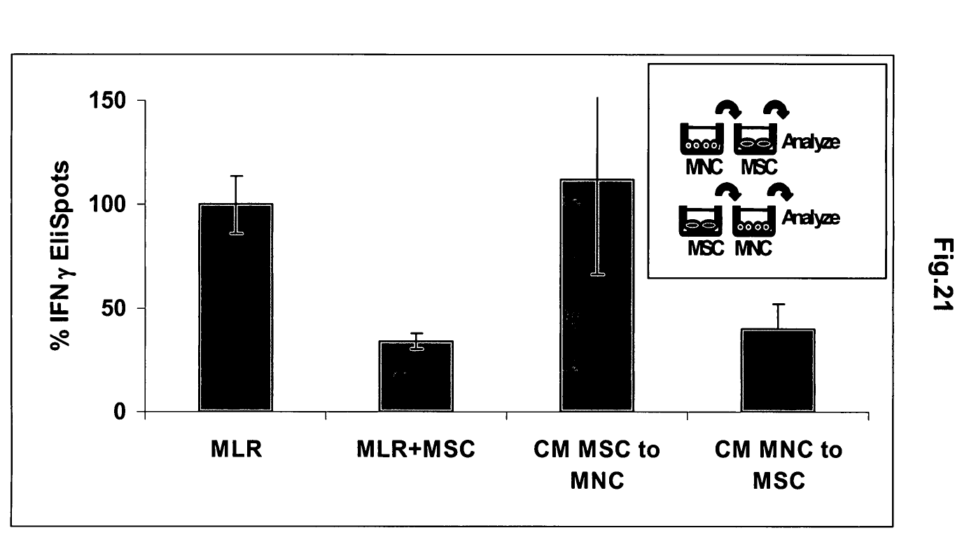

Cell free supernatant obtained from near confluent layers of human huMSC (Conditioned Medium, CM) had no inhibitory activity on allogeneic MLRs, and had stimulatory activity in some experiments. However MLR was significantly inhibited by addition of cell free CM obtained from near confluent layers of human huMSC mixed with human blood MNC (FIG. 21). This inhibitory effect was observed consistently when huMSC were pre-activated with blood MNC obtained either from same the donors as the MLR or unrelated third-party donor. We found that the huMSC and blood MNC CM became inhibitory within 12 hours and reached maximal potency by 24 hours (data not shown). Furthermore this activation step was not contact dependent since conditioned medium from huMSC co-cultured directly with third-party MNC had comparable inhibition to conditioned medium harvested from the cultures separated by a trans-well system. To examine further the activation process of huMSC by blood MNC, cell free supernatants were sequentially transferred from individual cultures of huMSC and blood MNC as shown in FIG. 21. Culture medium was incubated for 24 hours in each condition and the final supernatant was tested on an allogeneic MLR EliSpot-IFN-γ assay. Inhibitory activity was observed when the medium was conditioned first by the blood MNC and then the huMSC, but not with the reverse sequence (FIG. 21) confirming a necessary activation step of huMSC by soluble factors generated by blood MNC.

### EXAMPLE 17

### Blood CD14⁺ Monocytes Activate huMSC to Secrete Soluble Immunosuppressive Factor(s)

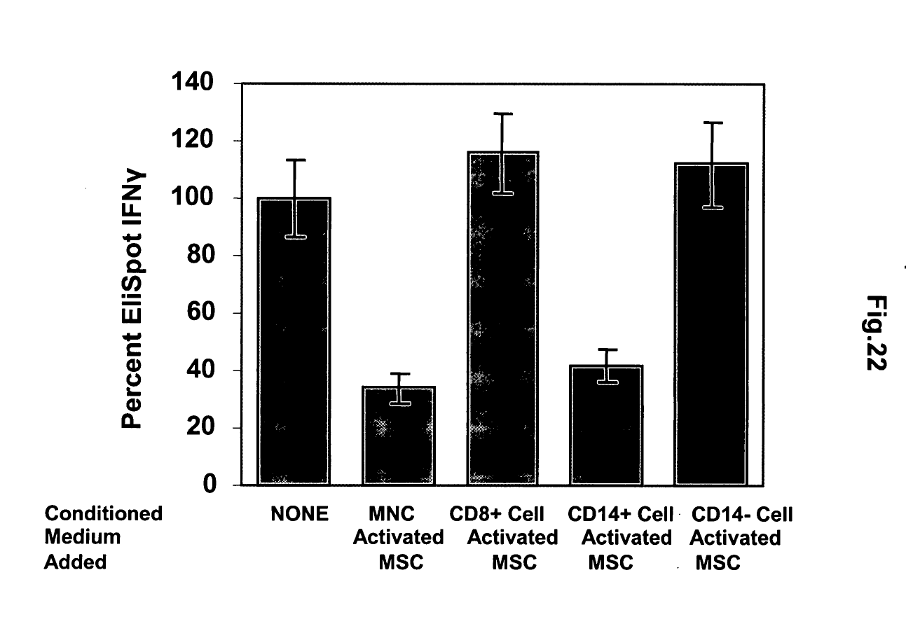

Next, applicants determined the subpopulation of cells in blood responsible for huMSC activation. huMSC were co-cultured with enriched populations of CD8⁺, CD14⁺, and CD19⁺ cells separately for 24 hours. Cell-free supernatants from these cultures were tested for their ability to inhibit alloreactivity using EliSpot-IFN-γ assay. Only the supernatant from huMSC and CD14⁺ cell co-culture inhibited the T-cell activation (FIG. 22). Furthermore, when the CD14 negative fraction of the blood cells were analyzed in the same fashion no inhibitory effect was observed.

### EXAMPLE 18

### The Role of TGFβ in huMSC-Mediated Inhibition of Alloreactivity

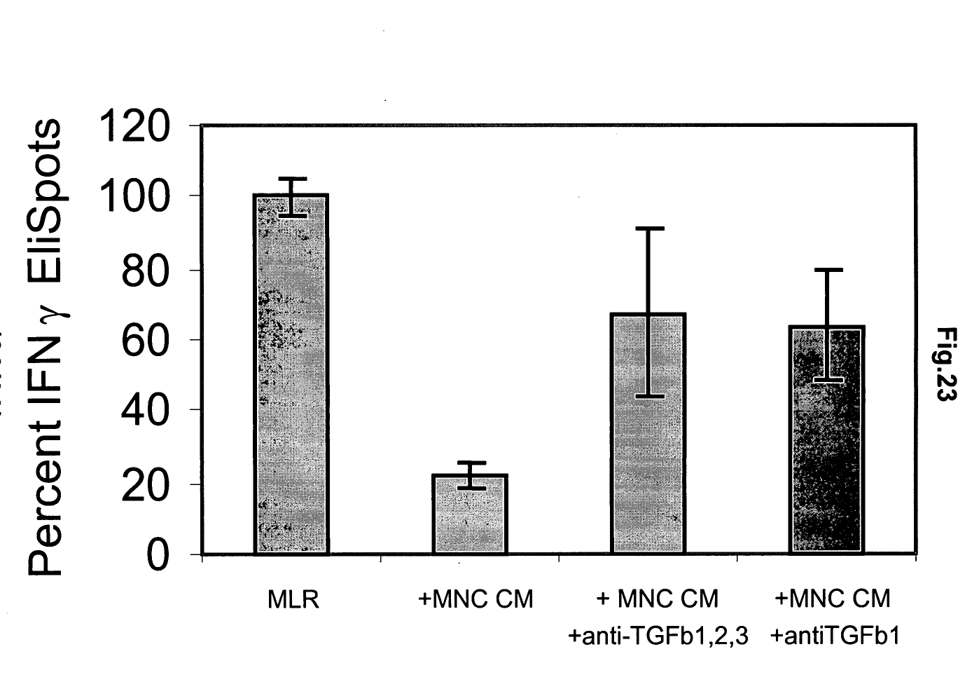

The effect of neutralizing anti-TGFβ antibodies was determined in allogeneic MLRs performed in the presence of cell-free conditioned culture medium of activated huMSC. We detected immunosuppressive cytokines such as TGFβ1, Hepatocyte Growth Factor (HGF) and Activin A in huMSC conditioned medium. There was either no change (HGF and Activin A) or only an additive increase (TGFβ1) in the concentration of these cytokines when huMSC were co-cultured with blood MNC. In particular, TGFβ1 concentrations were in the range of 0.3-0.6 ng/ml even in huMSC and blood MNC co-cultures and it was mostly in protein bound form (biologically inactive). These concentrations of TGFβ1 are at the low end of the concentrations we found to be inhibitory in allogeneic MLRs. In order to further determine if these low concentrations of TGFβ1 had a role in MSC mediated inhibition of alloreactivity, we used neutralizing antibodies to TGFβ1 (at ND₅₀ concentrations). The first antibody we used neutralized all isoforms of TGFβ (β1, β2, and β3, clone 1D11) and the second was specific to the β1 isoform only (clone 9016.1). We found a significant amelioration of the huMSC-mediated inhibition of alloreactivity using both antibodies, suggesting a role for TGFβ1 in this process (FIG. 23). We did not detect human IL-10 in culture supernatants of MSCs but there was a significant induction of IL-10 in co-cultures of huMSC and blood MNC compared to MNC alone. However, addition of IL-10 receptor antibodies did not reverse the inhibition of MLR mediated either by MSCs or by activated MSC culture conditioned medium. These data, combined with published data on immunosuppressive properties of huMSC, provide a compelling rationale to study huMSC as a potential therapeutic strategy to potentially facilitate allogeneic CD133⁺ EPC vasculogenic functional response to ischemia, as well the potential to ameliorate allogeneic immune reactivity.

### EXAMPLE 19

### Measurement of Secreted Factors and mRNA Expression in Hypoxic Conditions

To assess the effects of an ischemic environment on CD133 and MNC, cells will be subjected to both hypoxic (5% O₂) and normoxic (21% O₂) conditions to mimic the ischemic model. To confirm secretion studies utilizing CBA shown in our preliminary data, supernatants collected from cells will be analyzed in triplicate for production of angiogenic factors (IL-8, VEGF, TNF) using ELISA assay (BD Biosciences, Chemicon International). We will then isolate mRNA from the cultured cells using Trizol reagent to compare expression of key factors and receptors under the influence of hypoxic conditions as compared to normal conditions. Isolated mRNA will be checked for purity. We will probe for VEGFR2, CXCR1, CXCR2, CXCR4 (SDF1 receptor), TNF-α, and TGFβ1. RT-PCR analyses will be performed in the Gene Expression Array Core Facility (GEACF) at Case Western Reserve University.

### EXPERIMENT 20

### Generation of huMSC Conditioned Medium for Use in Mitogenic, Chemotaxis and Other Assays

HuMSC will be cultured and conditioned medium will be generated using 1×105 huMSC plated into 35 mm dishes in growth medium to permit attachment. 12 hours later the growth medium will be removed and cells will be washed before incubating in serum-free medium for 24 hours to eliminate stimulation by serum proteins in cellular cytoplasm and to allow for synchronization of huMSC cell cycle. huMSC will be washed again before fresh serum-free defined medium (SFDM) is added to cultures. huMSC will be permitted to condition the SFDM for 72 hours, after which time the medium will be removed, centrifuged to remove cellular debris, and normalized to cell number by dilution to a volume representative of 1×105 cells/ml.

### EXPERIMENT 21

### Establishment of Human Immune Cells in NOD.SCID Murine Model

Approximately 6-8 week old NOD.SCID/β2m−/− mice will be injected with 10⁶ lentiviral transduced adult blood mononuclear cells (AB-MNC) in 300 uL sterile saline. Mice will be imaged every 10 minutes for the first hour, every hour for the first 6 and every 12 h thereafter until engraftment is achieved. Optimal femoral artery ligation time will be defined by the time the cells reside in the lungs but before trafficking to other organs.

### EXPERIMENT 22

### Hind Limb Injury NOD.SCID Study Model

Femoral ligation will be used in NOD.SCID mice to establish a murine hind-limb injury model as described in the previous examples. After femoral artery ligation, the mice are randomized in to one of five groups. Group 1, control, is treated with intravenous injection of Clonetics media (0.02 ml). Group 2 is treated with intravenous injection of selected CD133 (106 in 0.02 ml). Group 3 animals receive third passage human MSC (106 in 0.02 ml). Group 4 animals receive CD133 and huMSC (106+106 in 0.02 ml). Group 5 animals receive CD133+ huMSC+ conditioned media from huMSC. The animals are survived for four weeks. The blood flow measurements on both feet will be repeated at 30 minutes, 7 days, 14 days and 28 days after the surgery. Delineation of what cell population is contributing to neoangiogenesis will include FISH analyses for Y chromosome, SH3 and SH4, CD31+ and P1H12+ in situ, using confocal immunofluorescence to delineate EPC and huMSC. At the end of study, the mice will be sacrificed. The tissue samples from lower calf muscle of both ischemic and healthy hind limbs will be harvested for fresh frozen (liquid nitrogen) and formalin fixation. Frozen sections of 6 μm thickness will be mounted on saline-coated glass slides, and stained using immunohistochemistry technique to identify incorporation of human cells by staining with anti-human CD31 antibody. The extent of neovascularization will be assessed by measuring capillary density in paraffin embedded sections (×40 magnification). These sections are stained for alkaline phosphatase with indoxyl-tetrazolium and counterstained with eosin to detect capillary endothelial cells. If homing of GFP-expressing CD133+ cells are observed in the hindlimb injury, leg tissue from NOD.SCID mice will be isolated and used for LCM as described in the previous examples. RT-PCR will be utilized to screen for labeled cells expressing smooth muscle and vascular RNA.

1. A method of inducing neovascularization in a subject in need thereof, the method comprising administering to the subject a therapeutically effective amount of a composition comprising conditioned cell culture medium from a first population of cells comprising (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof.

2. The method of claim 1, wherein the first population of cells is derived from umbilical cord blood.

3. The method of claim 2, wherein the umbilical cord blood is from a single umbilical cord.

4. The method of claim 2, wherein the first population of cells is derived from a umbilical cord blood from a plurality of umbilical cord.

5. The method of claim 1, wherein the AC133+ are genetically modified.

6. The method of claim 5, wherein the cells are genetically modified to express a transgene.

7-8. (canceled)

9. The method of claim 1, wherein at least 5% of the cells in the first population are (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof.

10-22. (canceled)

23. The method of claim 1, where the first population of cells, the second population of cells, or both, are cultured under hypoxic conditions.

24-39. (canceled)

40. The method of claim 1, wherein the mammal is afflicted with ischemia.

41. The method of claim 40, wherein the ischemia is selected from the group consisting of limb ischemia, ischemic cardiomyopathy, myocardial ischemia, cerebrovascular ischemia, renal ischemia, pulmonary ischemia and intestinal ischemia.

42. The method of claim 1, wherein the composition is administered to the subject via intracoronary, intravenous, intradermal, intraarterial, intramuscular, intracardiac, intraorbital, intraspinal or subcutaneous injection.

43-57. (canceled)

58. The method of claim 1, further comprising administering to the subject a therapeutically effective amount of therapeutic cells.

59. The method of claim 58, wherein the therapeutic cells are selected from the group consisting of(i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof.

60-61. (canceled)

62. A composition for inducing neovascularization in a subject, comprising conditioned cell culture medium from a first population of cells comprising (i) AC133+ cells; (ii) endothelial precursor cells; (iii) MAPCs; (iv) mesenchymal stem cells; (v) AC133-CD34-CD73-cells; (vi) CD34+ cells; or (vii) combinations thereof.

63. The composition of claim 62, wherein the first population of cells comprises AC133+ cells.

64. The composition of claim 62, wherein the first population of cells is derived from umbilical cord blood.

65. The composition of claim 64, wherein the umbilical cord blood is from a single umbilical cord.

66. The composition of claim 64, wherein the first population of cells is derived from a umbilical cord blood from a plurality of umbilical cords.

67-104. (canceled)

105. An implantable device comprising the compositions of claim 62.

106. The implantable device of claim 105, wherein the implantable device is a sustained release device.

107. The implantable device of claim 105, wherein the implantable device comprises a matrix.

108-114. (canceled)

115. A method for distributing the composition of claims 62 for use by health care professionals, the method comprising placing the composition into a package under sterile conditions and distributing the package for use by health care professionals.

116. (canceled)

117. A method of providing a composition for use by health care professionals for the treatment of a disorder in a subject, the method comprising:
(a) providing a sample of umbilical cord blood;
(b) culturing at least one cell from the umbilical cord blood in a cell culture medium to generate conditioned media;
(c) concentrating the protein components of the conditioned media and formulating a pharmaceutical composition which comprises at least one component;
(d) packaging the composition under sterile conditions; and
(e) distributing the package for use by health care professionals in treating the disorder in the subject.

118. The method of claim 117, wherein the sample is a cryopreserved sample.

119. The method of claim 117, wherein the umbilical cord blood is autologous to the subject.

120. The method of claim 117, wherein the umbilical cord blood is allogenic to the subject.

121-144. (canceled)
